(19) United States
(12) Patent Application Publication
(10) Pub. No.: US 2023/0346789 A1
(43) Pub. Date: Nov. 2, 2023

(54) METHODS OF TREATING ENHANCING BRAIN TUMORS USING COMBINATION THERAPY

(71) Applicants: MSD International GmbH, LUZERN (CH); MSD International Business GmbH, LUZERN (CH); Servier Pharmaceuticals LLC., BOSTON, MA (US)

(72) Inventors: Patrick WEN, Lexington, MA (US); Tim CLOUGHESY, CALABASAS, CA (US); Islam HASSAN, NATICK, MA (US)

(21) Appl. No.: 18/193,958

(22) Filed: Mar. 31, 2023

Related U.S. Application Data
(60) Provisional application No. 63/325,991, filed on Mar. 31, 2022.

(51) Int. Cl. A61K 31/53; A61K 39/395

(52) U.S. Cl. CPC A61K 31/53; A61K 39/3955

(57) ABSTRACT

Provided are methods for treating enhancing brain tumors in patients in need thereof using a combination of an inhibitor of mutant IDH1/2 enzyme and an anti PD-1 agent.

### CROSS-REFERENCE TO RELATED APPLICATIONS

This application claims priority to U.S. Provisional Application Ser. No. 63/325,991, filed Mar. 31, 2022, which is incorporated herein by reference in its entirety.

### FIELD

Provided herein are combination therapies comprising an IDH1 inhibitor and an anti-PD1 antibody for treating enhancing brain tumors.

### BACKGROUND

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid.

The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (DEC-2008) to UniProtKB; Kullmann et al., Submitted (JUN-1996) to the EMBL/GenBank/DDBJ databases; and Sjoeblom et al., Science 314:268-274(2006).

Non-mutant, e.g., wild type, IDH1 catalyzes the oxidative decarboxylation of isocitrate to α-ketoglutarate thereby reducing NAD⁺ (NADP⁺) to NADH (NADPH), e.g., in the forward reaction:

Isocitrate+NAD⁺(NADP⁺)→α-KG+CO₂+NADH(NADPH)+H⁺.

It has been discovered that mutations of IDH1 present in certain cancer cells result in a new ability of the enzyme to catalyze the NADPH-dependent reduction of α-ketoglutarate to R(−)-2-hydroxyglutarate (2HG). The production of R(−)-2-hydroxyglutarate (2HG) is believed to contribute to the formation and progression of cancer (Dang, L et al., Nature 2009, 462:739-44).

IDH2 (isocitrate dehydrogenase 2 (NADP⁺), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_002168.2 and NP_002159.2 respectively. The nucleotide and amino acid sequence for human IDH2 are also described in, e.g., Huh et al., Submitted (NOV-1992) to the EMBL/GenBank/DDBJ databases; and The MGC Project Team, Genome Res. 14:2121-2127(2004).

Non-mutant, e.g., wild type, IDH2 catalyzes the oxidative decarboxylation of isocitrate to α-ketoglutarate (α-KG) thereby reducing NAD⁺ (NADP⁺) to NADH (NADPH), e.g., in the forward reaction:

Isocitrate+NAD⁺(NADP⁺)→α-KG+CO₂+NADH(NADPH)+H⁺.

It has been discovered that mutations of IDH2 present in certain cancer cells result in a new ability of the enzyme to catalyze the NADPH-dependent reduction of α-ketoglutarate to R(−)-2-hydroxyglutarate (2HG). R(−)-2-hydroxyglutarate (2HG) is not formed by wild-type IDH2. The production of R(−)-2-hydroxyglutarate (2HG) is believed to contribute to the formation and progression of cancer (Dang, L et al, Nature 2009, 462:739-44). The inhibition of mutant IDH1 and/or mutant IDH2 and their neoactivity is therefore a potential therapeutic treatment for cancer.

Oligodendrogliomas and astrocytomas are primary brain tumors and represent subtypes of gliomas (e.g., adult-type diffuse gliomas). Traditionally, oligodendrogliomas and astrocytomas have been classified in accordance with their histopathological phenotypes, but more recent classification guidelines have moved toward an increased role of molecular markers in CNS tumor taxonomy. Per 2021 WHO (World Health Organization) classification of CNS tumors (WHO CNS5), adult-type diffuse gliomas are classified into three subtypes: oligodendroglioma, astrocytoma and glioblastoma. Oligodendrogliomas harbor IDH1 or IDH2 mutations and show loss of 1p19q. Per CNS5, oligodendrogliomas occur in grades 2 or 3. Astrocytomas harbor IDH1 or IDH2 mutations but no loss of 1p19q. Per CNS5, astrocytomas occur in grades 2, 3 or 4. Finally, glioblastomas are characterized by a lack of IDH mutations (i.e., wild-type IDH). (Louis, D. N et al, Neuro-Oncology, 23:1231-1251 (2021).

Contrast enhancement is an imaging presentation of primary brain tumors, including IDH mutant gliomas, on MRI (Magnetic Resonance Imaging) or CT (Computerized Tomography) scans. IDH mutant grade 2/3 gliomas usually start as a non-enhancing tumor, which means they do not take up an intravenously infused contrasting agent. The lack of contrast enhancement indicates the blood brain barrier is largely intact. The appearance of contrast-enhancement, meaning the contrast agent is getting into the brain, is an imaging feature that is generally associated with aggressive tumor biology/higher grade tumors, compared to non-contrast enhancing disease which tends to be associated with lower grade tumors. The appearance of contrast enhancement is an important factor that is considered when determining the need for radiation and/or chemotherapy.

Recurrent or progressive enhancing IDH-mutant glioma are oligodendrogliomas and astrocytomas that harbor IDH1 or IDH2 gene mutation and have recurred or progressed after receiving standard of care therapy including surgery, radiation and chemotherapy.

Vorasidenib (AG-881) is an orally available, brain penetrant second-generation dual mutant isocitrate dehydrogenase 1 and 2 (mIDH1/2) inhibitor currently undergoing clinical trials for the treatment of non-enhancing glioma, including low grade glioma.

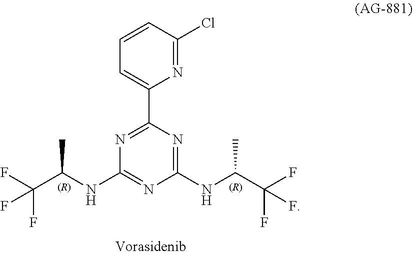

Vorasidenib (6-(6-chloropyridin-2-yl)-N²,N⁴-bis((R)-1,1,1-trifluoropropan-2-yl)-1,3,5-triazine-2,4-diamine) is disclosed in U.S. Pat. No. 9,579,324, which is incorporated herein by reference in its entirety.

In a phase I dose escalation study (clinical trial NCT02481154, Mellinghoff, I K et al, “Vorasidenib, a Dual Inhibitor of Mutant IDH1/2, in Recurrent or Progressive Glioma; Results of a First-in-Human Phase I Trial” Clin Cancer Res 15 Aug. 2021; 27 (16): 4491-4499) vorasidenib showed clinical activity and prolonged disease control in patients with non-enhancing gliomas. In contrast, only limited efficacy was observed with vorasidenib monotherapy in patients with recurrent enhancing gliomas.

Thus, there remains a significant unmet need for methods of treating enhancing IDH-mutant gliomas (e.g., oligodendrogliomas and astrocytomas).

### SUMMARY

In one embodiment, provided is a method of treating an enhancing brain tumor in a patient in need thereof comprising administering to the patient a therapeutically effective amount of vorasidenib or a pharmaceutically acceptable salt, hydrate, solvate, or cocrystal thereof, and pembrolizumab.

In one embodiment, provided is a method of treating an enhancing glioma in a patient in need thereof comprising administering to the patient a therapeutically effective amount of vorasidenib or a pharmaceutically acceptable salt, hydrate, solvate, or cocrystal thereof, and pembrolizumab.

In some embodiments, the enhancing brain tumor (e.g., glioma) is recurrent or progressive.

In some embodiments, the enhancing brain tumor (e.g., glioma) is an oligodendroglioma or astrocytoma.

In some embodiments, the enhancing brain tumor is an astrocytoma.

In some embodiments, the enhancing brain tumor (e.g., glioma) is characterized by the presence of an isocitrate dehydrogenase (IDH) mutation.

In some embodiments, the enhancing brain tumor (e.g., glioma) is characterized by the presence of an IDH1 mutation, wherein the IDH1 mutation results in accumulation of R(−)-2-hydroxyglutarate in a patient.

In some embodiments, the IDH1 mutation is an R132X mutation.

In some embodiments, the IDH1 mutation is an R132H or R132C mutation.

In some embodiments, the enhancing brain tumor (e.g., glioma) is characterized by the presence of an IDH2 mutation, wherein the IDH2 mutation results in accumulation of R(−)-2-hydroxyglutarate in a patient.

In some embodiments, the IDH2 mutation is an R140X mutation.

In some embodiments, the IDH2 mutation is an R140Q, R140W, or R140L mutation.

In some embodiments, the IDH2 mutation is an R172X mutation.

In some embodiments, the IDH2 mutation is an R172K or R172G mutation.

In some embodiments, the enhancing brain tumor (e.g., glioma) is characterized by the presence of an IDH1 mutation and an IDH2 mutation, wherein the IDH1 and IDH2 mutations collectively result in accumulation of R(−)-2-hydroxyglutarate in a patient.

In some embodiments, vorasidenib is administered in non-salt form (i.e., as a free base).

In some embodiments, vorasidenib is administered as a cocrystal.

In some embodiments, vorasidenib is administered as a cocrystal with citric acid.

In some embodiments, vorasidenib is administered at a dose between about 10 mg/day and about 100 mg/day.

In some embodiments, vorasidenib is administered at a dose between about 10 mg/day and about 50 mg/day.

In some embodiments, vorasidenib is administered at a dose between about 20 mg/day and about 40 mg/day.

In some embodiments, vorasidenib is administered at a dose of about 10 mg/day, about 15 mg/day, about 20 mg/day, about 25 mg/day, about 30 mg/day, about 35 mg/day, about 40 mg/day, about 45 mg/day or about 50 mg/day.

In some embodiments, vorasidenib is administered at a dose of about 10 mg/day, about 20 mg/day or about 40 mg/day.

In some embodiments, vorasidenib is administered at a dose of about 20 mg/day or about 40 mg/day.

In some embodiments, vorasidenib is administered at a dose of about 40 mg/day.

In some embodiments, vorasidenib is administered once or twice daily.

In some embodiments, vorasidenib is administered once daily.

In some embodiments, vorasidenib is administered at a dose between about 10 mg and about 100 mg once daily.

In some embodiments, vorasidenib is administered at a dose between about 10 mg and about 50 mg once daily.

In some embodiments, vorasidenib is administered at a dose between about 20 mg and about 40 mg once daily.

In some embodiments, vorasidenib is administered at a dose of about 10 mg, about 15 mg, about 20 mg, about 25 mg, about 30 mg, about 35 mg, about 40 mg, about 45 mg or about 50 mg once daily.

In some embodiments, vorasidenib is administered at a dose of about 10 mg, about 20 mg or about 40 mg once daily.

In some embodiments, vorasidenib is administered at a dose of about 20 mg or about 40 mg once daily.

In some embodiments, vorasidenib is administered at a dose of about 40 mg once daily.

In some embodiments, pembrolizumab is administered at a dose between about 10 mg and about 500 mg every three weeks (Q3W) or every 6 weeks (Q6W).

In some embodiments, pembrolizumab is administered at a dose between about 100 mg and about 400 mg Q3W or Q6W.

In some embodiments, pembrolizumab is administered at a dose between about 200 mg and about 400 mg Q3W or Q6W.

In some embodiments, pembrolizumab is administered at a dose of about 100 mg, about 150 mg, about 200 mg, about 250 mg, about 300 mg, about 350 mg, about 400 mg, about 450 mg or about 500 mg Q3W or Q6W.

In some embodiments, pembrolizumab is administered at a dose of about 100 mg, about 200 mg or about 400 mg Q3W or Q6W.

In some embodiments, pembrolizumab is administered at a dose of about 200 mg or about 400 mg Q3W or Q6W.

In some embodiments, pembrolizumab is administered Q3W.

In some embodiments, pembrolizumab is administered Q6W.

In some embodiments, pembrolizumab is administered at a dose between about 100 mg and about 300 mg Q3W.

In some embodiments, pembrolizumab is administered at a dose of about 100 mg, 200 mg or 300 mg Q3W.

In some embodiments, pembrolizumab is administered at a dose of about 200 mg Q3W.

In some embodiments, pembrolizumab is administered at a dose between about 200 mg and about 500 mg Q3W.

In some embodiments, pembrolizumab is administered at a dose of about 200 mg, 400 mg or 500 mg Q3W.

In some embodiments, pembrolizumab is administered at a dose of about 400 mg Q3W.

In some embodiments, vorasidenib and pembrolizumab are administered sequentially.

In some embodiments, vorasidenib and pembrolizumab are administered concurrently.

### BRIEF DESCRIPTION OF THE DRAWINGS

FIG. 1—Design of the safety lead-in phase of a clinical trial for the combination of vorasidenib and pembrolizumab in patients with enhancing astrocytoma.

FIG. 2—Design of the randomized peri-operative phase of a clinical trial for the combination of vorasidenib and pembrolizumab in patients with enhancing astrocytoma.

### DETAILED DESCRIPTION

The details of construction and the arrangement of components set forth in the following description or illustrated in the drawings are not meant to be limiting. Other embodiments and different ways to practice the invention are expressly included. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Definitions

The terms “enhancing brain tumor” (e.g., enhancing glioma (e.g., enhancing astrocytoma, enhancing oligodendroglioma)) and “contrast-enhancing brain tumor” (e.g., contrast-enhancing glioma (e.g., contrast-enhancing astrocytoma, contrast-enhancing oligodendroglioma)) are used interchangeably herein to refer to brain tumors that show uptake of intravenous contrast agent in imaging studies (e.g., computerized tomography (CT) and magnetic resonance imaging (MRI)).

The term a “mutant IDH1 inhibitor” or “inhibitor of IDH1 mutant(s)” means a molecule e.g., a polypeptide, peptide, or small molecule (e.g., a molecule of less than 1,000 daltons), or aptomer, that binds to an IDH1 mutant subunit and inhibits neoactivity, e.g., by inhibiting formation of a dimer, e.g., a homodimer of mutant IDH1 subunits or a heterodimer of a mutant and a wildype subunit. The term a “mutant IDH2 inhibitor” or “inhibitor of IDH2 mutant(s)” means a molecule e.g., a polypeptide, peptide, or small molecule (e.g., a molecule of less than 1,000 daltons), or aptomer, that binds to an IDH2 mutant subunit and inhibits neoactivity, e.g., by inhibiting formation of a dimer, e.g., a homodimer of mutant IDH2 subunits or a heterodimer of a mutant and a wildype subunit. The term a “mutant IDH1/2 inhibitor” or “inhibitor of IDH1/2 mutant(s)” means a molecule e.g., a polypeptide, peptide, or small molecule (e.g., a molecule of less than 1,000 daltons), or aptomer, that binds to both an IDH1 mutant and an IDH2 mutant subunit and inhibits neoactivity. In some embodiments, the neoactivity inhibition is at least about 60%, 70%, 80%, 90%, 95% or 99% as compared to the activity in the absence of the mutant IDH1 inhibitor. In one embodiment, the mutant IDH1/2 inhibitor is vorasidenib.

The term “elevated levels of 2HG” means 10%, 20%, 30%, 50%, 75%, 100%, 200%, 500% or more 2HG is present in a subject that carries a mutant IDH allele than is present in a subject that does not carry a mutant IDH allele. The term “elevated levels of 2HG” may refer to the amount of 2HG within a cell, within a tumor, within an organ comprising a tumor, or within a bodily fluid.

The term “bodily fluid” includes one or more of amniotic fluid surrounding a fetus, aqueous humour, blood (e.g., blood plasma), serum, Cerebrospinal fluid, cerumen, chyme, Cowper's fluid, female ejaculate, interstitial fluid, lymph, breast milk, mucus (e.g., nasal drainage or phlegm), pleural fluid, pus, saliva, sebum, semen, serum, sweat, tears, urine, vaginal secretion, or vomit.

The terms “inhibit” or “prevent” include both complete and partial inhibition and prevention. An inhibitor may completely or partially inhibit the intended target.

The term “subject” is intended to include human and non-human animals. Exemplary human subjects include a human patient (referred to as a patient) having a disorder, e.g., a disorder described herein or a normal subject. The term “non-human animals” of one aspect of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc. In some embodiments, the subject is an adult subject, meaning a subject that is aged 18 years or older.

The term “treat” means decrease, suppress, attenuate, diminish, arrest, or stabilize the development or progression of a disease/disorder (e.g., a glioma (including adult diffuse gliomas such as oligodendroglioma and astrocytoma) characterized by the presence of a mutant allele of IDH1), lessen the severity of the disease/disorder or improve the symptoms associated with the disease/disorder.

An amount of a compound, including a pharmaceutically acceptable salt, solvate, cocrystal, tautomer, stereoisomer, isotopologue, prodrug or a polymorph thereof, effective to treat a disorder, or a “therapeutically effective amount” or “therapeutically effective dose” refers to an amount of the compound, including a pharmaceutically acceptable salt, solvate, tautomer, stereoisomer, isotopologue, prodrug, or a polymorph thereof, which is effective, upon single or multiple dose administration to a subject, in treating a cell, or in curing, alleviating, relieving or improving a subject with a disorder beyond that expected in the absence of such treatment.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals. When not administered as a fixed combination in one dosage unit form, the two compounds are administered independently at the same time or separately within time intervals that allow that the combination partners show a beneficial effect.

The term “co-administering” as used herein with respect to vorasidenib and pembrolizumab means that the vorasidenib and pembrolizumab may be administered together as part of a single dosage form (such as a composition comprising vorasidenib and pembrolizumab) or as separate, multiple dosage forms. Alternatively, pembrolizumab may be administered prior to, consecutively with, or following the administration of vorasidenib. In such combination therapy treatment, both vorasidenib and pembrolizumab are administered by conventional methods. The administration of a composition comprising both vorasidenib and pembrolizumab, to a subject does not preclude the separate administration of either vorasidenib or pembrolizumab to said subject at another time during a course of treatment. The term “co-administering” as used herein with respect to pembrolizumab means that pembrolizumab treatment may occur prior to, consecutively with, concurrently with or following the administration of vorasidenib. When not administered as a fixed combination in one dosage unit form, the two compounds are administered independently at the same time or separately within time intervals that allow that the combination partners show a beneficial effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (i.e., in separate dosage units, for example, separate tablets, e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the agents/drugs are administered to the same patient as part of the same course of therapy. The agents may be administered at the same point in time or immediately following one another. The agents may be administered in any order. The agents may be administered separately at different times during the course of therapy in such time intervals that the combination therapy is effective in treating cancer. In any case, the treatment regimen will provide beneficial effects in treating the conditions or disorders described herein.

The term “crystalline” refers to a solid having a highly regular chemical structure. In particular, a crystalline vorasidenib may be produced as one or more single crystalline forms of vorasidenib. For the purposes of this application, the terms “crystalline form”, “single crystalline form” and “polymorph” are synonymous; the terms distinguish between crystals that have different properties (e.g., different XRPD patterns and/or different DSC scan results). The term “polymorph” includes pseudopolymorphs, which are typically different solvates of a material, and thus their properties differ from one another. Thus, each distinct polymorph and pseudopolymorph of vorasidenib is considered to be a distinct single crystalline form herein. The term “substantially crystalline” refers to forms that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to a vorasidenib that is at least 70% crystalline. In other embodiments, substantially crystalline refers to a vorasidenib that is at least 90% crystalline.

The term “isolated” refers to forms that may be at least a particular weight percent of a particular crystalline form of compound. Particular weight percentages are 90%, 91%, 92%, 93%, 94%, 95%, 96%, 97%, 98%, 99%, 99.5%, 99.9%, or any percentage between 90% and 100%. The term “solvate or solvated” means a physical association of a compound, including a crystalline form thereof, of this invention with one or more solvent molecules. This physical association includes hydrogen bonding. In certain instances, the solvate will be capable of isolation, for example when one or more solvent molecules are incorporated in the crystal lattice of the crystalline solid. “Solvate or solvated” encompasses both solution-phase and isolable solvates. Representative solvates include, for example, a hydrate, ethanolates or a methanolate. The term “hydrate” is a solvate wherein the solvent molecule is H₂O that is present in a defined stoichiometric amount, and may, for example, include hemihydrate, monohydrate, dihydrate, or trihydrate.

The term “pharmaceutically acceptable carrier, adjuvant, or vehicle” refers to a carrier, adjuvant, or vehicle that may be administered to a subject, together with the active ingredient (e.g., vorasidenib, or a pharmaceutically acceptable salt, hydrate, solvate or cocrystal thereof or pembrolizumab), and which does not destroy the pharmacological activity thereof and is nontoxic when administered in doses sufficient to deliver a therapeutic amount of the active ingredient. Pharmaceutically acceptable carriers, adjuvants and vehicles that may be used in the pharmaceutical compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the active ingredients.

The term “a pharmaceutically-acceptable salt” as used herein refers to non-toxic acid or base addition salts of the compound to which the term refers. Examples of pharmaceutically acceptable salts are discussed in Berge et al., 1977, “Pharmaceutically Acceptable Salts.” J. Pharm. Sci. Vol. 66, pp. 1-19.

The term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms for oral administration include pills, tablets, capsules or the like in the case of solid compositions. Typical unit dosage forms for injectable (e.g., intravenous) administration include single-dose vials.

### Compounds

Vorasidenib as used herein refers to a compound of formula (I):

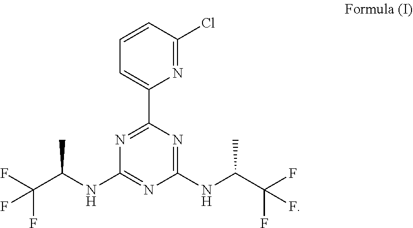 [SMILES: C[C@@H](Nc1nc(N[C@H](C)C(F)(F)F)nc(-c2cccc(Cl)n2)n1)C(F)(F)F]

Vorasidenib is also known as 6-(6-chloropyridin-2-yl)-N²,N⁴-bis((R)-1,1,1-trifluoropropan-2-yl)-1,3,5-triazine-2,4-diamine or AG-881.

The compound of formula (I) can be prepared by the method described in paragraphs [1032]-[1036] of U.S. Publication No. 2015/0018328 A1, which paragraphs are incorporated herein by reference.

As used herein, the terms “compound” and “pharmaceutically acceptable salt,” when referring to vorasidenib and pharmaceutically acceptable salts thereof, include vorasidenib and pharmaceutically acceptable salts in any form, including any tautomer or rotamer thereof, any solid form thereof (including any polymorphic form thereof), any solvate or hydrate form thereof, any cocrystal thereof, and any solution thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” of the vorasidenib includes any non-toxic salt that, upon administration to a recipient, is capable of providing, either directly or indirectly, vorasidenib. Pharmaceutically acceptable salts are described in detail in S. M. Berge, et al., J. Pharmaceutical Sciences, 1977, 66, 1-19, incorporated herein by reference.

As used herein, the term “cocrystal” refers to a crystalline solid made up of two or more neutral chemical species in a defined stoichiometric ratio that possesses distinct crystallographic and spectroscopic properties when compared to the species individually. A “cocrystal” is distinct from a “salt,” which is made up of charged-balanced charged species. The species making up a cocrystal typically are linked by hydrogen bonding and other non-covalent and non-ionic interactions. Thus, a pharmaceutical cocrystal of a drug typically comprises the drug and one or more coformers. Cocrystals of vorasidenib (e.g., cocrystals of vorasidenib and citric acid) have been described in U.S. Publication No. 2021/0198234 A1.

In some embodiments, vorasidenib is administered in non-salt form (i.e., free base form). In some embodiments, vorasidenib is administered as a cocrystal (e.g., a cocrystal with citric acid).

Vorasidenib may also comprise one or more isotopic substitutions. For example, H may be in any isotopic form (“Isotopologues”), including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like. For example, vorasidenib is enriched in a specific isotopic form of H, C and/or O by at least about 60%, 65%, 70%, 75%, 80%, 85%, 90%, 95%, 96%, 97%, 98%, or 99%.

Pembrolizumab (Keytruda®) as used herein is a programmed death receptor-1 (PD1)-blocking antibody. Pembrolizumab is produced in recombinant Chinese hamster ovary (CHO) cells. Pembrolizumab is a potent humanized immunoglobulin G4 (IgG4) monoclonal antibody (mAb) with high specificity of binding to the programmed cell death 1 (PD1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD1. Pembrolizumab has an approximate molecular weight of 149 kDa. Pembrolizumab has an acceptable preclinical safety profile and is in clinical development as an intravenous (IV) immunotherapy for advanced malignancies. Keytruda® (pembrolizumab) is indicated for the treatment of patients across a number of indications.

### Compositions and Routes of Administration

In one embodiment, the compounds utilized in the methods provided herein (i.e., vorasidenib or a pharmaceutically acceptable salt, solvate, hydrate or cocrystal thereof, and pembrolizumab) may be formulated with a pharmaceutically acceptable carrier or adjuvant into pharmaceutically acceptable compositions prior to being administered to a subject. In one embodiment, vorasidenib and pembrolizumab are formulated as one composition. In another embodiment, vorasidenib and pembrolizumab are formulated as separate compositions.

In another embodiment, such pharmaceutically acceptable compositions further comprise additional therapeutic agents in amounts effective for achieving a modulation of disease or disease symptoms, including those described herein.

The pharmaceutical compositions may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

In one embodiment, pembrolizumab is formulated as a sterile injectable preparation in water, further containing L-histidine, polysorbate and sucrose.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase and combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing the compound of formula (I), or a pharmaceutically acceptable salt thereof, with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions may be administered topically to the skin. The pharmaceutical composition should be formulated with a suitable ointment containing the active components suspended or dissolved in a carrier. Carriers for topical administration of the compounds of one aspect of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of one aspect of this invention may also be topically applied to the lower intestinal tract by rectal suppository formulation or in a suitable enema formulation. Topically-transdermal patches are also included in one aspect of this invention.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The amount of active ingredient that may be combined with the carrier materials to produce a single dosage form will vary depending upon the patient treated and the particular mode of administration. A typical preparation will contain from about 5% to about 95% active compound (w/w). Alternatively, such preparations contain from about 20% to about 80% active compound. In one embodiment, provided are dosage forms comprising the pharmaceutical compositions described herein. In one embodiment, the dosage form (e.g., the dosage form for vorasidenib) is an oral dosage form. In one embodiment, the dosage form for vorasidenib is a tablet or a capsule. In one embodiment, the dosage form for vorasidenib is a tablet. In one embodiment the dosage form for vorasidenib is a capsule.

In one embodiment, the dosage form for vorasidenib comprises between about 1 mg and about 500 mg of vorasidenib. In one embodiment, the dosage form comprises between about 1 mg and about 200 mg vorasidenib. In one embodiment, the dosage form comprises between about 1 mg and about 150 mg vorasidenib. In one embodiment, the dosage form comprises between about 1 mg and about 100 mg vorasidenib. In one embodiment, the dosage form comprises between about 1 mg and about 90 mg vorasidenib. In one embodiment, the dosage form comprises between about 1 mg and about 80 mg vorasidenib. In one embodiment, the dosage form comprises between about 1 mg and about 70 mg vorasidenib. In one embodiment, the dosage form comprises between about 1 mg and about 60 mg vorasidenib. In one embodiment, the dosage form comprises between about 1 mg and about 50 mg vorasidenib. In one embodiment, the dosage form comprises between about 1 mg and about 40 mg vorasidenib. In one embodiment, the dosage form comprises between about 1 mg and about 30 mg vorasidenib. In one embodiment, the dosage form comprises between about 1 mg and about 20 mg vorasidenib. In one embodiment, the dosage form comprises between about 1 mg and about 10 mg vorasidenib. In one embodiment, the dosage form comprises between about 1 mg and about 5 mg vorasidenib. In one embodiment, the dosage form comprises between about 5 mg and about 200 mg vorasidenib. In one embodiment, the dosage form comprises between about 5 mg and about 150 mg vorasidenib. In one embodiment, the dosage form comprises between about 5 mg and about 100 mg vorasidenib. In one embodiment, the dosage form comprises between about 5 mg and about 90 mg vorasidenib. In one embodiment, the dosage form comprises between about 5 mg and about 80 mg vorasidenib. In one embodiment, the dosage form comprises between about 5 mg and about 70 mg vorasidenib. In one embodiment, the dosage form comprises between about 5 mg and about 60 mg vorasidenib. In one embodiment, the dosage form comprises between about 5 mg and about 50 mg vorasidenib. In one embodiment, the dosage form comprises between about 5 mg and about 40 mg vorasidenib. In one embodiment, the dosage form comprises between about 5 mg and about 30 mg vorasidenib. In one embodiment, the dosage form comprises between about 5 mg and about 20 mg vorasidenib. In one embodiment, the dosage form comprises between about 5 mg and about 10 mg vorasidenib. In one embodiment, the dosage form comprises between about 10 mg and about 200 mg vorasidenib. In one embodiment, the dosage form comprises between about 10 mg and about 150 mg vorasidenib. In one embodiment, the dosage form comprises between about 10 mg and about 100 mg vorasidenib. In one embodiment, the dosage form comprises between about 10 mg and about 90 mg vorasidenib. In one embodiment, the dosage form comprises between about 10 mg and about 80 mg vorasidenib. In one embodiment, the dosage form comprises between about 10 mg and about 70 mg vorasidenib. In one embodiment, the dosage form comprises between about 10 mg and about 60 mg vorasidenib. In one embodiment, the dosage form comprises between about 10 mg and about 50 mg vorasidenib. In one embodiment, the dosage form comprises between about 10 mg and about 40 mg vorasidenib. In one embodiment, the dosage form comprises between about 10 mg and about 30 mg vorasidenib. In one embodiment, the dosage form comprises between about 10 mg and about 20 mg vorasidenib. In one embodiment, the dosage form comprises between about 20 mg and about 200 mg vorasidenib. In one embodiment, the dosage form comprises between about 20 mg and about 150 mg vorasidenib. In one embodiment, the dosage form comprises between about 20 mg and about 100 mg vorasidenib. In one embodiment, the dosage form comprises between about 20 mg and about 90 mg vorasidenib. In one embodiment, the dosage form comprises between about 20 mg and about 80 mg vorasidenib. In one embodiment, the dosage form comprises between about 20 mg and about 70 mg vorasidenib. In one embodiment, the dosage form comprises between about 20 mg and about 60 mg vorasidenib. In one embodiment, the dosage form comprises between about 20 mg and about 50 mg vorasidenib. In one embodiment, the dosage form comprises between about 20 mg and about 40 mg vorasidenib. In one embodiment, the dosage form comprises between about 20 mg and about 30 mg vorasidenib. In one embodiment, the dosage form comprises between about 30 mg and about 200 mg vorasidenib. In one embodiment, the dosage form comprises between about 30 mg and about 150 mg vorasidenib. In one embodiment, the dosage form comprises between about 30 mg and about 100 mg vorasidenib. In one embodiment, the dosage form comprises between about 30 mg and about 90 mg vorasidenib. In one embodiment, the dosage form comprises between about 30 mg and about 80 mg vorasidenib. In one embodiment, the dosage form comprises between about 30 mg and about 70 mg vorasidenib. In one embodiment, the dosage form comprises between about 30 mg and about 60 mg vorasidenib. In one embodiment, the dosage form comprises between about 30 mg and about 50 mg vorasidenib. In one embodiment, the dosage form comprises between about 30 mg and about 40 mg vorasidenib. In one embodiment, the dosage form comprises between about 40 mg and about 200 mg vorasidenib. In one embodiment, the dosage form comprises between about 40 mg and about 150 mg vorasidenib. In one embodiment, the dosage form comprises between about 40 mg and about 100 mg vorasidenib. In one embodiment, the dosage form comprises between about 40 mg and about 90 mg vorasidenib. In one embodiment, the dosage form comprises between about 40 mg and about 80 mg vorasidenib. In one embodiment, the dosage form comprises between about 40 mg and about 70 mg vorasidenib. In one embodiment, the dosage form comprises between about 40 mg and about 60 mg vorasidenib. In one embodiment, the dosage form comprises between about 40 mg and about 50 mg vorasidenib. In one embodiment, the dosage form comprises between about 50 mg and about 200 mg vorasidenib. In one embodiment, the dosage form comprises between about 50 mg and about 150 mg vorasidenib. In one embodiment, the dosage form comprises between about 50 mg and about 100 mg vorasidenib. In one embodiment, the dosage form comprises between about 50 mg and about 90 mg vorasidenib. In one embodiment, the dosage form comprises between about 50 mg and about 80 mg vorasidenib. In one embodiment, the dosage form comprises between about 50 mg and about 70 mg vorasidenib. In one embodiment, the dosage form comprises between about 50 mg and about 60 mg vorasidenib. In one embodiment, the dosage form comprises between about 60 mg and about 200 mg vorasidenib. In one embodiment, the dosage form comprises between about 60 mg and about 150 mg vorasidenib. In one embodiment, the dosage form comprises between about 60 mg and about 100 mg vorasidenib. In one embodiment, the dosage form comprises between about 60 mg and about 90 mg vorasidenib. In one embodiment, the dosage form comprises between about 60 mg and about 80 mg vorasidenib. In one embodiment, the dosage form comprises between about 60 mg and about 70 mg vorasidenib. In one embodiment, the dosage form comprises between about 70 mg and about 200 mg vorasidenib. In one embodiment, the dosage form comprises between about 70 mg and about 150 mg vorasidenib. In one embodiment, the dosage form comprises between about 70 mg and about 100 mg vorasidenib. In one embodiment, the dosage form comprises between about 70 mg and about 90 mg vorasidenib. In one embodiment, the dosage form comprises between about 70 mg and about 80 mg vorasidenib. In one embodiment, the dosage form comprises between about 80 mg and about 200 mg vorasidenib. In one embodiment, the dosage form comprises between about 80 mg and about 150 mg vorasidenib. In one embodiment, the dosage form comprises between about 80 mg and about 100 mg vorasidenib. In one embodiment, the dosage form comprises between about 80 mg and about 90 mg vorasidenib. In one embodiment, the dosage form comprises between about 90 mg and about 200 mg vorasidenib. In one embodiment, the dosage form comprises between about 90 mg and about 150 mg vorasidenib. In one embodiment, the dosage form comprises between about 90 mg and about 100 mg vorasidenib. In one embodiment, the dosage form comprises between about 100 mg and about 200 mg vorasidenib. In one embodiment, the dosage form comprises between about 100 mg and about 150 mg vorasidenib.

In one embodiment, the dosage form comprises about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg or about 500 mg vorasidenib. In one embodiment, the dosage form comprises about 1 mg, about 2 mg, about 3 mg, about 4 mg, about 5 mg, about 6 mg, about 7 mg, about 8 mg, about 9 mg or about 10 mg vorasidenib. In one embodiment, the dosage form comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg vorasidenib. In one embodiment, the dosage form comprises about 10 mg, about 15 mg, about 20 mg, about 25 mg, about 30 mg, about 35 mg, about 40 mg, about 45 mg or about 50 mg vorasidenib.

In one embodiment, the dosage form comprises about 1 mg vorasidenib. In one embodiment, the dosage form comprises about 2 mg vorasidenib. In one embodiment, the dosage form comprises about 3 mg vorasidenib. In one embodiment, the dosage form comprises about 4 mg vorasidenib. In one embodiment, the dosage form comprises about 5 mg vorasidenib. In one embodiment, the dosage form comprises about 6 mg vorasidenib. In one embodiment, the dosage form comprises about 7 mg vorasidenib. In one embodiment, the dosage form comprises about 8 mg vorasidenib. In one embodiment, the dosage form comprises about 9 mg vorasidenib. In one embodiment, the dosage form comprises about 10 mg vorasidenib. In one embodiment, the dosage form comprises about 15 mg vorasidenib. In one embodiment, the dosage form comprises about 20 mg vorasidenib. In one embodiment, the dosage form comprises about 25 mg vorasidenib. In one embodiment, the dosage form comprises about 30 mg vorasidenib. In one embodiment, the dosage form comprises about 35 mg vorasidenib. In one embodiment, the dosage form comprises about 40 mg vorasidenib. In one embodiment, the dosage form comprises about 45 mg vorasidenib. In one embodiment, the dosage form comprises about 50 mg vorasidenib. In one embodiment, the dosage form comprises about 55 mg vorasidenib. In one embodiment, the dosage form comprises about 60 mg vorasidenib. In one embodiment, the dosage form comprises about 65 mg vorasidenib. In one embodiment, the dosage form comprises about 70 mg vorasidenib. In one embodiment, the dosage form comprises about 75 mg vorasidenib. In one embodiment, the dosage form comprises about 80 mg vorasidenib. In one embodiment, the dosage form comprises about 85 mg vorasidenib. In one embodiment, the dosage form comprises about 90 mg vorasidenib. In one embodiment, the dosage form comprises about 95 mg vorasidenib. In one embodiment, the dosage form comprises about 100 mg vorasidenib. In one embodiment, the dosage form comprises about 110 mg vorasidenib. In one embodiment, the dosage form comprises about 120 mg vorasidenib. In one embodiment, the dosage form comprises about 130 mg vorasidenib. In one embodiment, the dosage form comprises about 140 mg vorasidenib. In one embodiment, the dosage form comprises about 150 mg vorasidenib. In one embodiment, the dosage form comprises about 160 mg vorasidenib. In one embodiment, the dosage form comprises about 170 mg vorasidenib. In one embodiment, the dosage form comprises about 180 mg vorasidenib. In one embodiment, the dosage form comprises about 190 mg vorasidenib. In one embodiment, the dosage form comprises about 200 mg vorasidenib. In one embodiment, the dosage form comprises about 250 mg vorasidenib. In one embodiment, the dosage form comprises about 300 mg vorasidenib. In one embodiment, the dosage form comprises about 350 mg vorasidenib. In one embodiment, the dosage form comprises about 400 mg vorasidenib. In one embodiment, the dosage form comprises about 450 mg vorasidenib. In one embodiment, the dosage form comprises about 500 mg vorasidenib.

In one embodiment, the dosage form (e.g., the dosage form for pembrolizumab) is an injectable dosage form (e.g., an injectable dosage form suitable for intravenous administration). In one embodiment, the dosage form for pembrolizumab is a single-dose vial.

In one embodiment, the dosage form for pembrolizumab comprises between about 1 mg and about 500 mg of pembrolizumab. In one embodiment, the dosage form comprises between about 10 mg and about 200 mg pembrolizumab. In one embodiment, the dosage form comprises between about 10 mg and about 150 mg pembrolizumab. In one embodiment, the dosage form comprises between about 10 mg and about 100 mg pembrolizumab. In one embodiment, the dosage form comprises between about 10 mg and about 90 mg pembrolizumab. In one embodiment, the dosage form comprises between about 10 mg and about 80 mg pembrolizumab. In one embodiment, the dosage form comprises between about 10 mg and about 70 mg pembrolizumab. In one embodiment, the dosage form comprises between about 10 mg and about 60 mg pembrolizumab. In one embodiment, the dosage form comprises between about 10 mg and about 50 mg pembrolizumab. In one embodiment, the dosage form comprises between about 20 mg and about 200 mg pembrolizumab. In one embodiment, the dosage form comprises between about 20 mg and about 150 mg pembrolizumab. In one embodiment, the dosage form comprises between about 20 mg and about 100 mg pembrolizumab. In one embodiment, the dosage form comprises between about 20 mg and about 90 mg pembrolizumab. In one embodiment, the dosage form comprises between about 20 mg and about 80 mg pembrolizumab. In one embodiment, the dosage form comprises between about 20 mg and about 70 mg pembrolizumab. In one embodiment, the dosage form comprises between about 20 mg and about 60 mg pembrolizumab. In one embodiment, the dosage form comprises between about 20 mg and about 50 mg pembrolizumab. In one embodiment, the dosage form comprises between about 30 mg and about 200 mg pembrolizumab. In one embodiment, the dosage form comprises between about 30 mg and about 150 mg pembrolizumab. In one embodiment, the dosage form comprises between about 30 mg and about 100 mg pembrolizumab. In one embodiment, the dosage form comprises between about 30 mg and about 90 mg pembrolizumab. In one embodiment, the dosage form comprises between about 30 mg and about 80 mg pembrolizumab. In one embodiment, the dosage form comprises between about 30 mg and about 70 mg pembrolizumab. In one embodiment, the dosage form comprises between about 30 mg and about 60 mg pembrolizumab. In one embodiment, the dosage form comprises between about 30 mg and about 50 mg pembrolizumab. In one embodiment, the dosage form comprises between about 40 mg and about 200 mg pembrolizumab. In one embodiment, the dosage form comprises between about 40 mg and about 150 mg pembrolizumab. In one embodiment, the dosage form comprises between about 40 mg and about 100 mg pembrolizumab. In one embodiment, the dosage form comprises between about 40 mg and about 90 mg pembrolizumab. In one embodiment, the dosage form comprises between about 40 mg and about 80 mg pembrolizumab. In one embodiment, the dosage form comprises between about 40 mg and about 70 mg pembrolizumab. In one embodiment, the dosage form comprises between about 40 mg and about 60 mg pembrolizumab. In one embodiment, the dosage form comprises between about 40 mg and about 50 mg pembrolizumab. In one embodiment, the dosage form comprises between about 50 mg and about 200 mg pembrolizumab. In one embodiment, the dosage form comprises between about 50 mg and about 150 mg pembrolizumab. In one embodiment, the dosage form comprises between about 50 mg and about 100 mg pembrolizumab. In one embodiment, the dosage form comprises between about 50 mg and about 90 mg pembrolizumab. In one embodiment, the dosage form comprises between about 50 mg and about 80 mg pembrolizumab. In one embodiment, the dosage form comprises between about 50 mg and about 70 mg pembrolizumab. In one embodiment, the dosage form comprises between about 50 mg and about 60 mg pembrolizumab. In one embodiment, the dosage form comprises between about 60 mg and about 200 mg pembrolizumab. In one embodiment, the dosage form comprises between about 60 mg and about 150 mg pembrolizumab. In one embodiment, the dosage form comprises between about 60 mg and about 100 mg pembrolizumab. In one embodiment, the dosage form comprises between about 60 mg and about 90 mg pembrolizumab. In one embodiment, the dosage form comprises between about 60 mg and about 80 mg pembrolizumab. In one embodiment, the dosage form comprises between about 60 mg and about 70 mg pembrolizumab. In one embodiment, the dosage form comprises between about 70 mg and about 200 mg pembrolizumab. In one embodiment, the dosage form comprises between about 70 mg and about 150 mg pembrolizumab. In one embodiment, the dosage form comprises between about 70 mg and about 100 mg pembrolizumab. In one embodiment, the dosage form comprises between about 70 mg and about 90 mg pembrolizumab. In one embodiment, the dosage form comprises between about 70 mg and about 80 mg pembrolizumab. In one embodiment, the dosage form comprises between about 80 mg and about 200 mg pembrolizumab. In one embodiment, the dosage form comprises between about 80 mg and about 150 mg pembrolizumab. In one embodiment, the dosage form comprises between about 80 mg and about 100 mg pembrolizumab. In one embodiment, the dosage form comprises between about 80 mg and about 90 mg pembrolizumab. In one embodiment, the dosage form comprises between about 90 mg and about 200 mg pembrolizumab. In one embodiment, the dosage form comprises between about 90 mg and about 150 mg pembrolizumab. In one embodiment, the dosage form comprises between about 90 mg and about 100 mg pembrolizumab. In one embodiment, the dosage form comprises between about 100 mg and about 200 mg pembrolizumab. In one embodiment, the dosage form comprises between about 100 mg and about 150 mg pembrolizumab.

In one embodiment, the dosage form comprises about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg or about 500 mg pembrolizumab. In one embodiment, the dosage form comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg. about 100 mg, about 150 mg, or about 200 mg pembrolizumab. In one embodiment, the dosage form comprises about 10 mg, about 15 mg, about 20 mg, about 25 mg, about 30 mg, about 35 mg, about 40 mg, about 45 mg or about 50 mg pembrolizumab. In one embodiment, the dosage form comprises about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg or about 200 mg pembrolizumab.

In one embodiment, the dosage form comprises about 10 mg pembrolizumab. In one embodiment, the dosage form comprises about 15 mg pembrolizumab. In one embodiment, the dosage form comprises about 20 mg pembrolizumab. In one embodiment, the dosage form comprises about 25 mg pembrolizumab. In one embodiment, the dosage form comprises about 30 mg pembrolizumab. In one embodiment, the dosage form comprises about 35 mg pembrolizumab. In one embodiment, the dosage form comprises about 40 mg pembrolizumab. In one embodiment, the dosage form comprises about 45 mg pembrolizumab. In one embodiment, the dosage form comprises about 50 mg pembrolizumab. In one embodiment, the dosage form comprises about 55 mg pembrolizumab. In one embodiment, the dosage form comprises about 60 mg pembrolizumab. In one embodiment, the dosage form comprises about 65 mg pembrolizumab. In one embodiment, the dosage form comprises about 70 mg pembrolizumab. In one embodiment, the dosage form comprises about 75 mg pembrolizumab. In one embodiment, the dosage form comprises about 80 mg pembrolizumab. In one embodiment, the dosage form comprises about 85 mg pembrolizumab. In one embodiment, the dosage form comprises about 90 mg pembrolizumab. In one embodiment, the dosage form comprises about 95 mg pembrolizumab. In one embodiment, the dosage form comprises about 100 mg pembrolizumab. In one embodiment, the dosage form comprises about 110 mg pembrolizumab. In one embodiment, the dosage form comprises about 120 mg pembrolizumab. In one embodiment, the dosage form comprises about 130 mg pembrolizumab. In one embodiment, the dosage form comprises about 140 mg pembrolizumab. In one embodiment, the dosage form comprises about 150 mg pembrolizumab. In one embodiment, the dosage form comprises about 160 mg pembrolizumab. In one embodiment, the dosage form comprises about 170 mg pembrolizumab. In one embodiment, the dosage form comprises about 180 mg pembrolizumab. In one embodiment, the dosage form comprises about 190 mg pembrolizumab. In one embodiment, the dosage form comprises about 200 mg pembrolizumab. In one embodiment, the dosage form comprises about 250 mg pembrolizumab. In one embodiment, the dosage form comprises about 300 mg pembrolizumab. In one embodiment, the dosage form comprises about 350 mg pembrolizumab. In one embodiment, the dosage form comprises about 400 mg pembrolizumab. In one embodiment, the dosage form comprises about 450 mg pembrolizumab. In one embodiment, the dosage form comprises about 500 mg pembrolizumab.

In one embodiment, the dosage form is a combination comprising vorasidenib and pembrolizumab.

In one embodiment, the dosage form comprises between about 35 mg and about 45 mg vorasidenib and between about 175 mg to 225 mg pembrolizumab. In one embodiment, the dosage form comprises between about 30 mg and about 50 mg vorasidenib and between about 150 mg and about 250 mg pembrolizumab. In one embodiment, the dosage form comprises between about 25 mg and about 55 mg vorasidenib and between about 125 mg and about 275 mg pembrolizumab. In one embodiment, the dosage form comprises between about 20 mg and about 60 mg vorasidenib and between about 100 mg and about 300 mg pembrolizumab. In one embodiment, the dosage form comprises between about 15 mg and about 65 mg vorasidenib and between about 75 mg and about 325 mg pembrolizumab. In one embodiment, the dosage form comprises between about 10 mg and about 70 mg vorasidenib and between about 50 mg and about 350 mg pembrolizumab. In one embodiment, the dosage form comprises between about 5 mg and about 75 mg vorasidenib and between about 25 mg and about 375 mg pembrolizumab. In one embodiment, the dosage form comprises between about 1 mg and about 80 mg vorasidenib and between about 1 mg and about 400 mg pembrolizumab. In one embodiment, the dosage form comprises between about 1 mg and about 90 mg vorasidenib and between about 1 mg and about 425 mg pembrolizumab. In one embodiment, the dosage form comprises between about 1 mg and about 100 mg vorasidenib and between about 1 mg and about 450 mg pembrolizumab. In one embodiment, the dosage form comprises between about 1 mg and about 150 mg vorasidenib and between about 1 mg and about 450 mg pembrolizumab. In one embodiment, the dosage form comprises between about 1 mg and about 200 mg vorasidenib and between about 1 mg and about 475 mg pembrolizumab. In one embodiment, the dosage form comprises between about 1 mg and about 250 mg vorasidenib and between about 1 mg and about 500 mg pembrolizumab. In one embodiment, the dosage form comprises between about 1 mg and about 300 mg vorasidenib and between about 1 mg and about 500 mg pembrolizumab. In one embodiment, the dosage form comprises between about 1 mg and about 350 mg vorasidenib and between about 1 mg and about 500 mg pembrolizumab. In one embodiment, the dosage form comprises between about 1 mg and about 400 mg vorasidenib and between about 1 mg and about 500 mg pembrolizumab. In one embodiment, the dosage form comprises between about 1 mg and about 450 mg vorasidenib and between about 1 mg and about 500 mg pembrolizumab. In one embodiment, the dosage form comprises between about 1 mg and about 500 mg vorasidenib and between about 1 mg and about 500 mg pembrolizumab.

In one embodiment, the dosage form comprises about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg or about 500 mg vorasidenib and about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg or about 500 mg pembrolizumab.

In one embodiment, the dosage form comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg vorasidenib and about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg. about 100 mg, about 150 mg, or about 200 mg pembrolizumab.

In one embodiment, the dosage form comprises about 10 mg, about 15 mg, about 20 mg, about 25 mg, about 30 mg, about 35 mg, about 40 mg, about 45 mg or about 50 mg vorasidenib and about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg. about 100 mg, about 150 mg, or about 200 mg pembrolizumab.

In one embodiment, the dosage form comprises about 10 mg, about 15 mg, about 20 mg, about 25 mg, about 30 mg, about 35 mg, about 40 mg, about 45 mg or about 50 mg vorasidenib and about 10 mg, about 15 mg, about 20 mg, about 25 mg, about 30 mg, about 35 mg, about 40 mg, about 45 mg or about 50 mg pembrolizumab.

In one embodiment, the dosage form comprises about 10 mg, about 15 mg, about 20 mg, about 25 mg, about 30 mg, about 35 mg, about 40 mg, about 45 mg or about 50 mg vorasidenib and about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg or about 200 mg pembrolizumab.

In one embodiment, the dosage form comprises about 40 mg vorasidenib and about 200 mg pembrolizumab. In one embodiment, the dosage form comprises about 35 mg vorasidenib and about 200 mg pembrolizumab. In one embodiment, the dosage form comprises about 30 mg vorasidenib and about 200 mg pembrolizumab. In one embodiment, the dosage form comprises about 25 mg vorasidenib and about 200 mg pembrolizumab. In one embodiment, the dosage form comprises about 20 mg vorasidenib and about 200 mg pembrolizumab. In one embodiment, the dosage form comprises about 15 mg vorasidenib and about 200 mg pembrolizumab. In one embodiment, the dosage form comprises about 10 mg vorasidenib and about 200 mg pembrolizumab. In one embodiment, the dosage form comprises about 5 mg vorasidenib and about 200 mg pembrolizumab. In one embodiment, the dosage form comprises about 1 mg vorasidenib and about 200 mg pembrolizumab.

In one embodiment, the dosage form comprises about 45 mg vorasidenib and about 200 mg pembrolizumab. In one embodiment, the dosage form comprises about 50 mg vorasidenib and about 200 mg pembrolizumab. In one embodiment, the dosage form comprises about 55 mg vorasidenib and about 200 mg pembrolizumab. In one embodiment, the dosage form comprises about 60 mg vorasidenib and about 200 mg pembrolizumab. In one embodiment, the dosage form comprises about 65 mg vorasidenib and about 200 mg pembrolizumab. In one embodiment, the dosage form comprises about 70 mg vorasidenib and about 200 mg pembrolizumab. In one embodiment, the dosage form comprises about 75 mg vorasidenib and about 200 mg pembrolizumab. In one embodiment, the dosage form comprises about 80 mg vorasidenib and about 200 mg pembrolizumab. In one embodiment, the dosage form comprises about 85 mg vorasidenib and about 200 mg pembrolizumab. In one embodiment, the dosage form comprises about 90 mg vorasidenib and about 200 mg pembrolizumab. In one embodiment, the dosage form comprises about 55 mg vorasidenib and about 200 mg pembrolizumab. In one embodiment, the dosage form comprises about 100 mg vorasidenib and about 200 mg pembrolizumab. In one embodiment, the dosage form comprises about 150 mg vorasidenib and about 200 mg pembrolizumab. In one embodiment, the dosage form comprises about 200 mg vorasidenib and about 200 mg pembrolizumab. In one embodiment, the dosage form comprises about 250 mg vorasidenib and about 200 mg pembrolizumab. In one embodiment, the dosage form comprises about 300 mg vorasidenib and about 200 mg pembrolizumab. In one embodiment, the dosage form comprises about 400 mg vorasidenib and about 200 mg pembrolizumab. In one embodiment, the dosage form comprises about 450 mg vorasidenib and about 200 mg pembrolizumab. In one embodiment, the dosage form comprises about 500 mg vorasidenib and about 200 mg pembrolizumab.

In one embodiment, the dosage form comprises about 40 mg vorasidenib and about 190 mg pembrolizumab. In one embodiment, the dosage form comprises about 40 mg vorasidenib and about 180 mg pembrolizumab. In one embodiment, the dosage form comprises about 40 mg vorasidenib and about 170 mg pembrolizumab. In one embodiment, the dosage form comprises about 40 mg vorasidenib and about 160 mg pembrolizumab. In one embodiment, the dosage form comprises about 40 mg vorasidenib and about 150 mg pembrolizumab. In one embodiment, the dosage form comprises about 40 mg vorasidenib and about 140 mg pembrolizumab. In one embodiment, the dosage form comprises about 40 mg vorasidenib and about 130 mg pembrolizumab. In one embodiment, the dosage form comprises about 40 mg vorasidenib and about 120 mg pembrolizumab. In one embodiment, the dosage form comprises about 40 mg vorasidenib and about 110 mg pembrolizumab. In one embodiment, the dosage form comprises about 40 mg vorasidenib and about 100 mg pembrolizumab.

In one embodiment, the dosage form comprises about 40 mg vorasidenib and about 90 mg pembrolizumab. In one embodiment, the dosage form comprises about 40 mg vorasidenib and about 80 mg pembrolizumab. In one embodiment, the dosage form comprises about 40 mg vorasidenib and about 70 mg pembrolizumab. In one embodiment, the dosage form comprises about 40 mg vorasidenib and about 60 mg pembrolizumab. In one embodiment, the dosage form comprises about 40 mg vorasidenib and about 50 mg pembrolizumab. In one embodiment, the dosage form comprises about 40 mg vorasidenib and about 40 mg pembrolizumab. In one embodiment, the dosage form comprises about 40 mg vorasidenib and about 30 mg pembrolizumab. In one embodiment, the dosage form comprises about 40 mg vorasidenib and about 20 mg pembrolizumab. In one embodiment, the dosage form comprises about 40 mg vorasidenib and about 10 mg pembrolizumab. In one embodiment, the dosage form comprises about 40 mg vorasidenib and about 1 mg pembrolizumab.

In one embodiment, the dosage form comprises about 40 mg vorasidenib and about 250 mg pembrolizumab. In one embodiment, the dosage form comprises about 40 mg vorasidenib and about 300 mg pembrolizumab. In one embodiment, the dosage form comprises about 40 mg vorasidenib and about 350 mg pembrolizumab. In one embodiment, the dosage form comprises about 40 mg vorasidenib and about 400 mg pembrolizumab.

In one embodiment, the dosage form comprises about 20 mg vorasidenib and about 190 mg pembrolizumab. In one embodiment, the dosage form comprises about 20 mg vorasidenib and about 180 mg pembrolizumab. In one embodiment, the dosage form comprises about 20 mg vorasidenib and about 170 mg pembrolizumab. In one embodiment, the dosage form comprises about 20 mg vorasidenib and about 160 mg pembrolizumab. In one embodiment, the dosage form comprises about 20 mg vorasidenib and about 150 mg pembrolizumab. In one embodiment, the dosage form comprises about 20 mg vorasidenib and about 120 mg pembrolizumab. In one embodiment, the dosage form comprises about 20 mg vorasidenib and about 130 mg pembrolizumab. In one embodiment, the dosage form comprises about 20 mg vorasidenib and about 120 mg pembrolizumab. In one embodiment, the dosage form comprises about 20 mg vorasidenib and about 110 mg pembrolizumab. In one embodiment, the dosage form comprises about 20 mg vorasidenib and about 100 mg pembrolizumab.

In one embodiment, the dosage form comprises about 20 mg vorasidenib and about 90 mg pembrolizumab. In one embodiment, the dosage form comprises about 20 mg vorasidenib and about 80 mg pembrolizumab. In one embodiment, the dosage form comprises about 20 mg vorasidenib and about 70 mg pembrolizumab. In one embodiment, the dosage form comprises about 20 mg vorasidenib and about 60 mg pembrolizumab. In one embodiment, the dosage form comprises about 20 mg vorasidenib and about 50 mg pembrolizumab. In one embodiment, the dosage form comprises about 20 mg vorasidenib and about 40 mg pembrolizumab. In one embodiment, the dosage form comprises about 20 mg vorasidenib and about 30 mg pembrolizumab. In one embodiment, the dosage form comprises about 20 mg vorasidenib and about 20 mg pembrolizumab. In one embodiment, the dosage form comprises about 20 mg vorasidenib and about 10 mg pembrolizumab. In one embodiment, the dosage form comprises about 20 mg vorasidenib and about 1 mg pembrolizumab.

In one embodiment, the dosage form comprises about 20 mg vorasidenib and about 250 mg pembrolizumab. In one embodiment, the dosage form comprises about 20 mg vorasidenib and about 300 mg pembrolizumab. In one embodiment, the dosage form comprises about 20 mg vorasidenib and about 350 mg pembrolizumab. In one embodiment, the dosage form comprises about 20 mg vorasidenib and about 400 mg pembrolizumab.

### Dosage

### Vorasidenib

Vorasidenib can be administered at a dose between about 1 mg/day and about 1000 mg/day. Vorasidenib can be administered at a dose between about 1 mg and about 500 mg one, two, three or four times a day. In one embodiment, vorasidenib is administered one time a day (once a day, or QD). In one embodiment, vorasidenib is administered two times a day (twice daily, or BID). In one embodiment, vorasidenib is administered three times a day (TID). In one embodiment, vorasidenib is administered four times a day (QID).

In one embodiment, the dose is between about 1 mg/day and about 500 mg/day. In one embodiment, the dose is between about 1 mg/day and about 200 mg/day. In one embodiment, the dose is between about 1 mg/day and about 150 mg/day. In one embodiment, the dose is between about 1 mg/day and about 100 mg/day. In one embodiment, the dose is between about 1 mg/day and about 90 mg/day. In one embodiment, the dose is between about 1 mg/day and about 80 mg/day. In one embodiment, the dose is between about 1 mg/day and about 70 mg/day. In one embodiment, the dose is between about 1 mg/day and about 60 mg/day. In one embodiment, the dose is between about 1 mg/day and about 50 mg/day. In one embodiment, the dose is between about 1 mg/day and about 40 mg/day. In one embodiment, the dose is between about 1 mg/day and about 30 mg/day. In one embodiment, the dose is between about 1 mg/day and about 20 mg/day. In one embodiment, the dose is between about 1 mg/day and about 10 mg/day. In one embodiment, the dose is between about 1 mg/day and about 5 mg/day. In one embodiment, the dose is between about 5 mg/day and about 500 mg/day. In one embodiment, the dose is between about 5 mg/day and about 200 mg/day. In one embodiment, the dose is between about 5 mg/day and about 150 mg/day. In one embodiment, the dose is between about 5 mg/day and about 100 mg/day. In one embodiment, the dose is between about 5 mg/day and about 90 mg/day. In one embodiment, the dose is between about 5 mg/day and about 80 mg/day. In one embodiment, the dose is between about 5 mg/day and about 70 mg/day. In one embodiment, the dose is between about 5 mg/day and about 60 mg/day. In one embodiment, the dose is between about 5 mg/day and about 50 mg/day. In one embodiment, the dose is between about 5 mg/day and about 40 mg/day. In one embodiment, the dose is between about 5 mg/day and about 30 mg/day. In one embodiment, the dose is between about 5 mg/day and about 20 mg/day. In one embodiment, the dose is between about 5 mg/day and about 10 mg/day. In one embodiment, the dose is between about 10 mg/day and about 500 mg/day. In one embodiment, the dose is between about 10 mg/day and about 200 mg/day. In one embodiment, the dose is between about 10 mg/day and about 150 mg/day. In one embodiment, the dose is between about 10 mg/day and about 100 mg/day. In one embodiment, the dose is between about 10 mg/day and about 90 mg/day. In one embodiment, the dose is between about 10 mg/day and about 80 mg/day. In one embodiment, the dose is between about 10 mg/day and about 70 mg/day. In one embodiment, the dose is between about 10 mg/day and about 60 mg/day. In one embodiment, the dose is between about 10 mg/day and about 50 mg/day. In one embodiment, the dose is between about 10 mg/day and about 40 mg/day. In one embodiment, the dose is between about 10 mg/day and about 30 mg/day. In one embodiment, the dose is between about 10 mg/day and about 20 mg/day. In one embodiment, the dose is between about 20 mg/day and about 500 mg/day. In one embodiment, the dose is between about 20 mg/day and about 200 mg/day. In one embodiment, the dose is between about 20 mg/day and about 150 mg/day. In one embodiment, the dose is between about 20 mg/day and about 100 mg/day. In one embodiment, the dose is between about 20 mg/day and about 90 mg/day. In one embodiment, the dose is between about 20 mg/day and about 80 mg/day. In one embodiment, the dose is between about 20 mg/day and about 70 mg/day. In one embodiment, the dose is between about 20 mg/day and about 60 mg/day. In one embodiment, the dose is between about 20 mg/day and about 50 mg/day. In one embodiment, the dose is between about 20 mg/day and about 40 mg/day. In one embodiment, the dose is between about 20 mg/day and about 30 mg/day. In one embodiment, the dose is between about 30 mg/day and about 500 mg/day. In one embodiment, the dose is between about 30 mg/day and about 200 mg/day. In one embodiment, the dose is between about 30 mg/day and about 150 mg/day. In one embodiment, the dose is between about 30 mg/day and about 100 mg/day. In one embodiment, the dose is between about 30 mg/day and about 90 mg/day. In one embodiment, the dose is between about 30 mg/day and about 80 mg/day. In one embodiment, the dose is between about 30 mg/day and about 70 mg/day. In one embodiment, the dose is between about 30 mg/day and about 60 mg/day. In one embodiment, the dose is between about 30 mg/day and about 50 mg/day. In one embodiment, the dose is between about 30 mg/day and about 40 mg/day. In one embodiment, the dose is between about 40 mg/day and about 500 mg/day. In one embodiment, the dose is between about 40 mg/day and about 200 mg/day. In one embodiment, the dose is between about 40 mg/day and about 150 mg/day. In one embodiment, the dose is between about 40 mg/day and about 100 mg/day. In one embodiment, the dose is between about 40 mg/day and about 90 mg/day. In one embodiment, the dose is between about 40 mg/day and about 80 mg/day. In one embodiment, the dose is between about 40 mg/day and about 70 mg/day. In one embodiment, the dose is between about 40 mg/day and about 60 mg/day. In one embodiment, the dose is between about 40 mg/day and about 50 mg/day. In one embodiment, the dose is between about 50 mg/day and about 500 mg/day. In one embodiment, the dose is between about 50 mg/day and about 200 mg/day. In one embodiment, the dose is between about 50 mg/day and about 150 mg/day. In one embodiment, the dose is between about 50 mg/day and about 100 mg/day. In one embodiment, the dose is between about 50 mg/day and about 90 mg/day. In one embodiment, the dose is between about 50 mg/day and about 80 mg/day. In one embodiment, the dose is between about 50 mg/day and about 70 mg/day. In one embodiment, the dose is between about 50 mg/day and about 60 mg/day. In one embodiment, the dose is between about 60 mg/day and about 500 mg/day. In one embodiment, the dose is between about 60 mg/day and about 200 mg/day. In one embodiment, the dose is between about 60 mg/day and about 150 mg/day. In one embodiment, the dose is between about 60 mg/day and about 100 mg/day. In one embodiment, the dose is between about 60 mg/day and about 90 mg/day. In one embodiment, the dose is between about 60 mg/day and about 80 mg/day. In one embodiment, the dose is between about 60 mg/day and about 70 mg/day. In one embodiment, the dose is between about 70 mg/day and about 500 mg/day. In one embodiment, the dose is between about 70 mg/day and about 200 mg/day. In one embodiment, the dose is between about 70 mg/day and about 150 mg/day. In one embodiment, the dose is between about 70 mg/day and about 100 mg/day. In one embodiment, the dose is between about 70 mg/day and about 90 mg/day. In one embodiment, the dose is between about 70 mg/day and about 80 mg/day. In one embodiment, the dose is between about 80 mg/day and about 500 mg/day. In one embodiment, the dose is between about 80 mg/day and about 200 mg/day. In one embodiment, the dose is between about 80 mg/day and about 150 mg/day. In one embodiment, the dose is between about 80 mg/day and about 100 mg/day. In one embodiment, the dose is between about 80 mg/day and about 90 mg/day. In one embodiment, the dose is between about 90 mg/day and about 500 mg/day. In one embodiment, the dose is between about 90 mg/day and about 200 mg/day. In one embodiment, the dose is between about 90 mg/day and about 150 mg/day. In one embodiment, the dose is between about 90 mg/day and about 100 mg/day. In one embodiment, the dose is between about 100 mg/day and about 500 mg/day. In one embodiment, the dose is between about 100 mg/day and about 200 mg/day. In one embodiment, the dose is between about 100 mg/day and about 150 mg/day. In one embodiment, the dose is about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 250 mg/day, about 300 mg/day, about 350 mg/day, about 400 mg/day, about 450 mg/day, about 500 mg/day or about 1000 mg/day. In one embodiment, the dose is about 1 mg/day, about 2 mg/day, about 3 mg/day, about 4 mg/day, about 5 mg/day, about 6 mg/day, about 7 mg/day, about 8 mg/day, about 9 mg/day or about 10 mg/day. In one embodiment, the dose is about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day or about 100 mg/day. In one embodiment, the dose is about 10 mg/day, about 15 mg/day, about 20 mg/day, about 25 mg/day, about 30 mg/day, about 35 mg/day, about 40 mg/day, about 45 mg/day or about 50 mg/day.

In one embodiment, the dose is about 1 mg/day. In one embodiment, the dose is about 2 mg/day. In one embodiment, the dose is about 3 mg/day. In one embodiment, the dose is about 4 mg/day. In one embodiment, the dose is about 5 mg/day. In one embodiment, the dose is about 6 mg/day. In one embodiment, the dose is about 7 mg/day. In one embodiment, the dose is about 8 mg/day. In one embodiment, the dose is about 9 mg/day. In one embodiment, the dose is about 10 mg/day. In one embodiment, the dose is about 15 mg/day. In one embodiment, the dose is about 20 mg/day. In one embodiment, the dose is about 25 mg/day. In one embodiment, the dose is about 30 mg/day. In one embodiment, the dose is about 35 mg/day. In one embodiment, the dose is about 40 mg/day. In one embodiment, the dose is about 45 mg/day. In one embodiment, the dose is about 50 mg/day. In one embodiment, the dose is about 55 mg/day. In one embodiment, the dose is about 60 mg/day. In one embodiment, the dose is about 65 mg/day. In one embodiment, the dose is about 70 mg/day. In one embodiment, the dose is about 75 mg/day. In one embodiment, the dose is about 80 mg/day. In one embodiment, the dose is about 85 mg/day. In one embodiment, the dose is about 90 mg/day. In one embodiment, the dose is about 95 mg/day. In one embodiment, the dose is about 100 mg/day. In one embodiment, the dose is about 110 mg/day. In one embodiment, the dose is about 120 mg/day. In one embodiment, the dose is about 130 mg/day. In one embodiment, the dose is about 140 mg/day. In one embodiment, the dose is about 150 mg/day. In one embodiment, the dose is about 160 mg/day. In one embodiment, the dose is about 170 mg/day. In one embodiment, the dose is about 180 mg/day. In one embodiment, the dose is about 190 mg/day. In one embodiment, the dose is about 200 mg/day. In one embodiment, the dose is about 250 mg/day. In one embodiment, the dose is about 300 mg/day. In one embodiment, the dose is about 350 mg/day. In one embodiment, the dose is about 400 mg/day. In one embodiment, the dose is about 450 mg/day. In one embodiment, the dose is about 500 mg/day. In one embodiment, the dose is about 600 mg/day. In one embodiment, the dose is about 700 mg/day. In one embodiment, the dose is about 800 mg/day. In one embodiment, the dose is about 900 mg/day. In one embodiment, the dose is about 1000 mg/day.

In one embodiment, the dose is between about 1 mg and about 200 mg once daily. In one embodiment, the dose is between about 1 mg and about 150 mg once daily. In one embodiment, the dose is between about 1 mg and about 100 mg once daily. In one embodiment, the dose is between about 1 mg and about 90 mg once daily. In one embodiment, the dose is between about 1 mg and about 80 mg once daily. In one embodiment, the dose is between about 1 mg and about 70 mg once daily. In one embodiment, the dose is between about 1 mg and about 60 mg once daily. In one embodiment, the dose is between about 1 mg and about 50 mg once daily. In one embodiment, the dose is between about 1 mg and about 40 mg once daily. In one embodiment, the dose is between about 1 mg and about 30 mg once daily. In one embodiment, the dose is between about 1 mg and about 20 mg once daily. In one embodiment, the dose is between about 1 mg and about 10 mg once daily. In one embodiment, the dose is between about 1 mg and about 5 mg once daily. In one embodiment, the dose is between about 5 mg and about 200 mg once daily. In one embodiment, the dose is between about 5 mg and about 150 mg once daily. In one embodiment, the dose is between about 5 mg and about 100 mg once daily. In one embodiment, the dose is between about 5 mg and about 90 mg once daily. In one embodiment, the dose is between about 5 mg and about 80 mg once daily. In one embodiment, the dose is between about 5 mg and about 70 mg once daily. In one embodiment, the dose is between about 5 mg and about 60 mg once daily. In one embodiment, the dose is between about 5 mg and about 50 mg once daily. In one embodiment, the dose is between about 5 mg and about 40 mg once daily. In one embodiment, the dose is between about 5 mg and about 30 mg once daily. In one embodiment, the dose is between about 5 mg and about 20 mg once daily. In one embodiment, the dose is between about 5 mg and about 10 mg once daily. In one embodiment, the dose is between about 10 mg and about 200 mg once daily. In one embodiment, the dose is between about 10 mg and about 150 mg once daily. In one embodiment, the dose is between about 10 mg and about 100 mg once daily. In one embodiment, the dose is between about 10 mg and about 90 mg once daily. In one embodiment, the dose is between about 10 mg and about 80 mg once daily. In one embodiment, the dose is between about 10 mg and about 70 mg once daily. In one embodiment, the dose is between about 10 mg and about 60 mg once daily. In one embodiment, the dose is between about 10 mg and about 50 mg once daily. In one embodiment, the dose is between about 10 mg and about 40 mg once daily. In one embodiment, the dose is between about 10 mg and about 30 mg once daily. In one embodiment, the dose is between about 10 mg and about 20 mg once daily. In one embodiment, the dose is between about 20 mg and about 200 mg once daily. In one embodiment, the dose is between about 20 mg and about 150 mg once daily. In one embodiment, the dose is between about 20 mg and about 100 mg once daily. In one embodiment, the dose is between about 20 mg and about 90 mg once daily. In one embodiment, the dose is between about 20 mg and about 80 mg once daily. In one embodiment, the dose is between about 20 mg and about 70 mg once daily. In one embodiment, the dose is between about 20 mg and about 60 mg once daily. In one embodiment, the dose is between about 20 mg and about 50 mg once daily. In one embodiment, the dose is between about 20 mg and about 40 mg once daily. In one embodiment, the dose is between about 20 mg and about 30 mg once daily. In one embodiment, the dose is between about 30 mg and about 200 mg once daily. In one embodiment, the dose is between about 30 mg and about 150 mg once daily. In one embodiment, the dose is between about 30 mg and about 100 mg once daily. In one embodiment, the dose is between about 30 mg and about 90 mg once daily. In one embodiment, the dose is between about 30 mg and about 80 mg once daily. In one embodiment, the dose is between about 30 mg and about 70 mg once daily. In one embodiment, the dose is between about 30 mg and about 60 mg once daily. In one embodiment, the dose is between about 30 mg and about 50 mg once daily. In one embodiment, the dose is between about 30 mg and about 40 mg once daily. In one embodiment, the dose is between about 40 mg and about 200 mg once daily. In one embodiment, the dose is between about 40 mg and about 150 mg once daily. In one embodiment, the dose is between about 40 mg and about 100 mg once daily. In one embodiment, the dose is between about 40 mg and about 90 mg once daily. In one embodiment, the dose is between about 40 mg and about 80 mg once daily. In one embodiment, the dose is between about 40 mg and about 70 mg once daily. In one embodiment, the dose is between about 40 mg and about 60 mg once daily. In one embodiment, the dose is between about 40 mg and about 50 mg once daily. In one embodiment, the dose is between about 50 mg and about 200 mg once daily. In one embodiment, the dose is between about 50 mg and about 150 mg once daily. In one embodiment, the dose is between about 50 mg and about 100 mg once daily. In one embodiment, the dose is between about 50 mg and about 90 mg once daily. In one embodiment, the dose is between about 50 mg and about 80 mg once daily. In one embodiment, the dose is between about 50 mg and about 70 mg once daily. In one embodiment, the dose is between about 50 mg and about 60 mg once daily. In one embodiment, the dose is between about 60 mg and about 200 mg once daily. In one embodiment, the dose is between about 60 mg and about 150 mg once daily. In one embodiment, the dose is between about 60 mg and about 100 mg once daily. In one embodiment, the dose is between about 60 mg and about 90 mg once daily. In one embodiment, the dose is between about 60 mg and about 80 mg once daily. In one embodiment, the dose is between about 60 mg and about 70 mg once daily. In one embodiment, the dose is between about 70 mg and about 200 mg once daily. In one embodiment, the dose is between about 70 mg and about 150 mg once daily. In one embodiment, the dose is between about 70 mg and about 100 mg once daily. In one embodiment, the dose is between about 70 mg and about 90 mg once daily. In one embodiment, the dose is between about 70 mg and about 80 mg once daily. In one embodiment, the dose is between about 80 mg and about 200 mg once daily. In one embodiment, the dose is between about 80 mg and about 150 mg once daily. In one embodiment, the dose is between about 80 mg and about 100 mg once daily. In one embodiment, the dose is between about 80 mg and about 90 mg once daily. In one embodiment, the dose is between about 90 mg and about 200 mg once daily. In one embodiment, the dose is between about 90 mg and about 150 mg once daily. In one embodiment, the dose is between about 90 mg and about 100 mg once daily. In one embodiment, the dose is between about 100 mg and about 200 mg once daily. In one embodiment, the dose is between about 100 mg and about 150 mg once daily. In one embodiment, the dose is about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg or about 500 mg once daily. In one embodiment, the dose is about 1 mg, about 2 mg, about 3 mg, about 4 mg, about 5 mg, about 6 mg, about 7 mg, about 8 mg, about 9 mg or about 10 mg once daily. In one embodiment, the dose is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg once daily. In one embodiment, the dose is about 10 mg, about 15 mg, about 20 mg, about 25 mg, about 30 mg, about 35 mg, about 40 mg, about 45 mg or about 50 mg once daily.

In one embodiment, the dose is about 1 mg once daily. In one embodiment, the dose is about 2 mg once daily. In one embodiment, the dose is about 3 mg once daily. In one embodiment, the dose is about 4 mg once daily. In one embodiment, the dose is about 5 mg once daily. In one embodiment, the dose is about 6 mg once daily. In one embodiment, the dose is about 7 mg once daily. In one embodiment, the dose is about 8 mg once daily. In one embodiment, the dose is about 9 mg once daily. In one embodiment, the dose is about 10 mg once daily. In one embodiment, the dose is about 15 mg once daily. In one embodiment, the dose is about 20 mg once daily. In one embodiment, the dose is about 25 mg once daily. In one embodiment, the dose is about 30 mg once daily. In one embodiment, the dose is about 35 mg once daily. In one embodiment, the dose is about 40 mg once daily. In one embodiment, the dose is about 45 mg once daily. In one embodiment, the dose is about 50 mg once daily. In one embodiment, the dose is about 55 mg once daily. In one embodiment, the dose is about 60 mg once daily. In one embodiment, the dose is about 65 mg once daily. In one embodiment, the dose is about 70 mg once daily. In one embodiment, the dose is about 75 mg once daily. In one embodiment, the dose is about 80 mg once daily. In one embodiment, the dose is about 85 mg once daily. In one embodiment, the dose is about 90 mg once daily. In one embodiment, the dose is about 95 mg once daily. In one embodiment, the dose is about 100 mg once daily. In one embodiment, the dose is about 110 mg once daily. In one embodiment, the dose is about 120 mg once daily. In one embodiment, the dose is about 130 mg once daily. In one embodiment, the dose is about 140 mg once daily. In one embodiment, the dose is about 150 mg once daily. In one embodiment, the dose is about 160 mg once daily. In one embodiment, the dose is about 170 mg once daily. In one embodiment, the dose is about 180 mg once daily. In one embodiment, the dose is about 190 mg once daily. In one embodiment, the dose is about 200 mg once daily. In one embodiment, the dose is about 250 mg once daily. In one embodiment, the dose is about 300 mg once daily. In one embodiment, the dose is about 350 mg once daily. In one embodiment, the dose is about 400 mg once daily. In one embodiment, the dose is about 450 mg once daily. In one embodiment, the dose is about 500 mg once daily.

In one embodiment, the dose is between about 1 mg and about 200 mg twice daily. In one embodiment, the dose is between about 1 mg and about 150 mg twice daily. In one embodiment, the dose is between about 1 mg and about 100 mg twice daily. In one embodiment, the dose is between about 1 mg and about 90 mg twice daily. In one embodiment, the dose is between about 1 mg and about 80 mg twice daily. In one embodiment, the dose is between about 1 mg and about 70 mg twice daily. In one embodiment, the dose is between about 1 mg and about 60 mg twice daily. In one embodiment, the dose is between about 1 mg and about 50 mg twice daily. In one embodiment, the dose is between about 1 mg and about 40 mg twice daily. In one embodiment, the dose is between about 1 mg and about 30 mg twice daily. In one embodiment, the dose is between about 1 mg and about 20 mg twice daily. In one embodiment, the dose is between about 1 mg and about 10 mg twice daily. In one embodiment, the dose is between about 1 mg and about 5 mg twice daily. In one embodiment, the dose is between about 5 mg and about 200 mg twice daily. In one embodiment, the dose is between about 5 mg and about 150 mg twice daily. In one embodiment, the dose is between about 5 mg and about 100 mg twice daily. In one embodiment, the dose is between about 5 mg and about 90 mg twice daily. In one embodiment, the dose is between about 5 mg and about 80 mg twice daily. In one embodiment, the dose is between about 5 mg and about 70 mg twice daily. In one embodiment, the dose is between about 5 mg and about 60 mg twice daily. In one embodiment, the dose is between about 5 mg and about 50 mg twice daily. In one embodiment, the dose is between about 5 mg and about 40 mg twice daily. In one embodiment, the dose is between about 5 mg and about 30 mg twice daily. In one embodiment, the dose is between about 5 mg and about 20 mg twice daily. In one embodiment, the dose is between about 5 mg and about 10 mg twice daily. In one embodiment, the dose is between about 10 mg and about 200 mg twice daily. In one embodiment, the dose is between about 10 mg and about 150 mg twice daily. In one embodiment, the dose is between about 10 mg and about 100 mg twice daily. In one embodiment, the dose is between about 10 mg and about 90 mg twice daily. In one embodiment, the dose is between about 10 mg and about 80 mg twice daily. In one embodiment, the dose is between about 10 mg and about 70 mg twice daily. In one embodiment, the dose is between about 10 mg and about 60 mg twice daily. In one embodiment, the dose is between about 10 mg and about 50 mg twice daily. In one embodiment, the dose is between about 10 mg and about 40 mg twice daily. In one embodiment, the dose is between about 10 mg and about 30 mg twice daily. In one embodiment, the dose is between about 10 mg and about 20 mg twice daily. In one embodiment, the dose is between about 20 mg and about 200 mg twice daily. In one embodiment, the dose is between about 20 mg and about 150 mg twice daily. In one embodiment, the dose is between about 20 mg and about 100 mg twice daily. In one embodiment, the dose is between about 20 mg and about 90 mg twice daily. In one embodiment, the dose is between about 20 mg and about 80 mg twice daily. In one embodiment, the dose is between about 20 mg and about 70 mg twice daily. In one embodiment, the dose is between about 20 mg and about 60 mg twice daily. In one embodiment, the dose is between about 20 mg and about 50 mg twice daily. In one embodiment, the dose is between about 20 mg and about 40 mg twice daily. In one embodiment, the dose is between about 20 mg and about 30 mg twice daily. In one embodiment, the dose is between about 30 mg and about 200 mg twice daily. In one embodiment, the dose is between about 30 mg and about 150 mg twice daily. In one embodiment, the dose is between about 30 mg and about 100 mg twice daily. In one embodiment, the dose is between about 30 mg and about 90 mg twice daily. In one embodiment, the dose is between about 30 mg and about 80 mg twice daily. In one embodiment, the dose is between about 30 mg and about 70 mg twice daily. In one embodiment, the dose is between about 30 mg and about 60 mg twice daily. In one embodiment, the dose is between about 30 mg and about 50 mg twice daily. In one embodiment, the dose is between about 30 mg and about 40 mg twice daily. In one embodiment, the dose is between about 40 mg and about 200 mg twice daily. In one embodiment, the dose is between about 40 mg and about 150 mg twice daily. In one embodiment, the dose is between about 40 mg and about 100 mg twice daily. In one embodiment, the dose is between about 40 mg and about 90 mg twice daily. In one embodiment, the dose is between about 40 mg and about 80 mg twice daily. In one embodiment, the dose is between about 40 mg and about 70 mg twice daily. In one embodiment, the dose is between about 40 mg and about 60 mg twice daily. In one embodiment, the dose is between about 40 mg and about 50 mg twice daily. In one embodiment, the dose is between about 50 mg and about 200 mg twice daily. In one embodiment, the dose is between about 50 mg and about 150 mg twice daily. In one embodiment, the dose is between about 50 mg and about 100 mg twice daily. In one embodiment, the dose is between about 50 mg and about 90 mg twice daily. In one embodiment, the dose is between about 50 mg and about 80 mg twice daily. In one embodiment, the dose is between about 50 mg and about 70 mg twice daily. In one embodiment, the dose is between about 50 mg and about 60 mg twice daily. In one embodiment, the dose is between about 60 mg and about 200 mg twice daily. In one embodiment, the dose is between about 60 mg and about 150 mg twice daily. In one embodiment, the dose is between about 60 mg and about 100 mg twice daily. In one embodiment, the dose is between about 60 mg and about 90 mg twice daily. In one embodiment, the dose is between about 60 mg and about 80 mg twice daily. In one embodiment, the dose is between about 60 mg and about 70 mg twice daily. In one embodiment, the dose is between about 70 mg and about 200 mg twice daily. In one embodiment, the dose is between about 70 mg and about 150 mg twice daily. In one embodiment, the dose is between about 70 mg and about 100 mg twice daily. In one embodiment, the dose is between about 70 mg and about 90 mg twice daily. In one embodiment, the dose is between about 70 mg and about 80 mg twice daily. In one embodiment, the dose is between about 80 mg and about 200 mg twice daily. In one embodiment, the dose is between about 80 mg and about 150 mg twice daily. In one embodiment, the dose is between about 80 mg and about 100 mg twice daily. In one embodiment, the dose is between about 80 mg and about 90 mg twice daily. In one embodiment, the dose is between about 90 mg and about 200 mg twice daily. In one embodiment, the dose is between about 90 mg and about 150 mg twice daily. In one embodiment, the dose is between about 90 mg and about 100 mg twice daily. In one embodiment, the dose is between about 100 mg and about 200 mg twice daily. In one embodiment, the dose is between about 100 mg and about 150 mg twice daily. In one embodiment, the dose is about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg or about 500 mg twice daily. In one embodiment, the dose is about 1 mg, about 2 mg, about 3 mg, about 4 mg, about 5 mg, about 6 mg, about 7 mg, about 8 mg, about 9 mg or about 10 mg twice daily. In one embodiment, the dose is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg twice daily. In one embodiment, the dose is about 10 mg, about 15 mg, about 20 mg, about 25 mg, about 30 mg, about 35 mg, about 40 mg, about 45 mg or about 50 mg twice daily.

In one embodiment, the dose is about 1 mg twice daily. In one embodiment, the dose is about 2 mg twice daily. In one embodiment, the dose is about 3 mg twice daily. In one embodiment, the dose is about 4 mg twice daily. In one embodiment, the dose is about 5 mg twice daily. In one embodiment, the dose is about 6 mg twice daily. In one embodiment, the dose is about 7 mg twice daily. In one embodiment, the dose is about 8 mg twice daily. In one embodiment, the dose is about 9 mg twice daily. In one embodiment, the dose is about 10 mg twice daily. In one embodiment, the dose is about 15 mg twice daily. In one embodiment, the dose is about 20 mg twice daily. In one embodiment, the dose is about 25 mg twice daily. In one embodiment, the dose is about 30 mg twice daily. In one embodiment, the dose is about 35 mg twice daily. In one embodiment, the dose is about 40 mg twice daily. In one embodiment, the dose is about 45 mg twice daily. In one embodiment, the dose is about 50 mg twice daily. In one embodiment, the dose is about 55 mg twice daily. In one embodiment, the dose is about 60 mg twice daily. In one embodiment, the dose is about 65 mg twice daily. In one embodiment, the dose is about 70 mg twice daily. In one embodiment, the dose is about 75 mg twice daily. In one embodiment, the dose is about 80 mg twice daily. In one embodiment, the dose is about 85 mg twice daily. In one embodiment, the dose is about 90 mg twice daily. In one embodiment, the dose is about 95 mg twice daily. In one embodiment, the dose is about 100 mg twice daily. In one embodiment, the dose is about 110 mg twice daily. In one embodiment, the dose is about 120 mg twice daily. In one embodiment, the dose is about 130 mg twice daily. In one embodiment, the dose is about 140 mg twice daily. In one embodiment, the dose is about 150 mg twice daily. In one embodiment, the dose is about 160 mg twice daily. In one embodiment, the dose is about 170 mg twice daily. In one embodiment, the dose is about 180 mg twice daily. In one embodiment, the dose is about 190 mg twice daily. In one embodiment, the dose is about 200 mg twice daily. In one embodiment, the dose is about 250 mg twice daily. In one embodiment, the dose is about 300 mg twice daily. In one embodiment, the dose is about 350 mg twice daily. In one embodiment, the dose is about 400 mg twice daily. In one embodiment, the dose is about 450 mg twice daily. In one embodiment, the dose is about 500 mg twice daily.

### Pembrolizumab

In one embodiment, pembrolizumab is administered at doses between about 1 mg and about 500 mg at an interval between one and ten weeks. In one embodiment, the dose of pembrolizumab is between about 50 mg and about 500 mg. In one embodiment, the dose of pembrolizumab is between about 50 mg and about 400 mg. In one embodiment, the dose of pembrolizumab is between about 50 mg and about 300 mg. In one embodiment, the dose of pembrolizumab is between about 50 mg and about 200 mg. In one embodiment, the dose of pembrolizumab is between about 50 mg and about 100 mg. In one embodiment, the dose of pembrolizumab is between about 100 mg and about 500 mg. In one embodiment, the dose of pembrolizumab is between about 100 mg and about 400 mg. In one embodiment, the dose of pembrolizumab is between about 100 mg and about 300 mg. In one embodiment, the dose of pembrolizumab is between about 100 mg and about 200 mg. In one embodiment, the dose of pembrolizumab is between about 200 mg and about 500 mg. In one embodiment, the dose of pembrolizumab is between about 200 mg and about 400 mg. In one embodiment, the dose of pembrolizumab is between about 200 mg and about 300 mg.

In one embodiment, the dose of pembrolizumab is selected from about 50 mg, about 100 mg, about 150 mg, about 200 mg, about 250 mg, about 300 mg, about 350 mg, about 400 mg, about 450 mg and about 500 mg. In one embodiment, the dose of pembrolizumab is selected from about 50 mg, about 100 mg, about 150 mg, about 200 mg, about 250 mg, about 300 mg, about 350 mg and about 400 mg. In one embodiment, the dose of pembrolizumab is selected from about 200 mg and about 400 mg. In one embodiment, the dose of pembrolizumab is about 50 mg. In one embodiment, the dose of pembrolizumab is about 100 mg. In one embodiment, the dose of pembrolizumab is about 150 mg. In one embodiment, the dose of pembrolizumab is about 200 mg. In one embodiment, the dose of pembrolizumab is about 250 mg. In one embodiment, the dose of pembrolizumab is about 300 mg. In one embodiment, the dose of pembrolizumab is about 350 mg. In one embodiment, the dose of pembrolizumab is about 400 mg. In one embodiment, the dose of pembrolizumab is about 450 mg. In one embodiment, the dose of pembrolizumab is about 500 mg.

In one embodiment, the interval is one week (e.g., pembrolizumab is administered once a week). In one embodiment, the interval is two weeks (e.g., pembrolizumab is administered once every two weeks). In one embodiment, the interval is three weeks (e.g., pembrolizumab is administered once every three weeks). In one embodiment, the interval is four weeks (e.g., pembrolizumab is administered once every four weeks). In one embodiment, the interval is five weeks (e.g., pembrolizumab is administered once every five weeks). In one embodiment, the interval is six weeks (e.g., pembrolizumab is administered once every six weeks). In one embodiment, the interval is seven weeks (e.g., pembrolizumab is administered once every seven weeks). In one embodiment, the interval is eight weeks (e.g., pembrolizumab is administered once every eight weeks). In one embodiment, the interval is nine weeks (e.g., pembrolizumab is administered once every nine weeks). In one embodiment, the interval is ten weeks (e.g., pembrolizumab is administered once every ten weeks).

In one embodiment, the dose of pembrolizumab is between about 50 mg and about 500 mg Q3W. In one embodiment, the dose of pembrolizumab is between about 50 mg and about 400 mg Q3W. In one embodiment, the dose of pembrolizumab is between about 50 mg and about 300 mg Q3W. In one embodiment, the dose of pembrolizumab is between about 50 mg and about 200 mg Q3W. In one embodiment, the dose of pembrolizumab is between about 50 mg and about 100 mg Q3W. In one embodiment, the dose of pembrolizumab is between about 100 mg and about 500 mg Q3W. In one embodiment, the dose of pembrolizumab is between about 100 mg and about 400 mg Q3W. In one embodiment, the dose of pembrolizumab is between about 100 mg and about 300 mg Q3W. In one embodiment, the dose of pembrolizumab is between about 100 mg and about 200 mg Q3W. In one embodiment, the dose of pembrolizumab is between about 200 mg and about 500 mg Q3W. In one embodiment, the dose of pembrolizumab is between about 200 mg and about 400 mg Q3W. In one embodiment, the dose of pembrolizumab is between about 200 mg and about 300 mg Q3W.

In one embodiment, the dose of pembrolizumab is selected from about 50 mg, about 100 mg, about 150 mg, about 200 mg, about 250 mg, about 300 mg, about 350 mg, about 400 mg, about 450 mg and about 500 mg Q3W. In one embodiment, the dose of pembrolizumab is selected from about 50 mg, about 100 mg, about 150 mg, about 200 mg, about 250 mg, about 300 mg, about 350 mg and about 400 mg Q3W. In one embodiment, the dose of pembrolizumab is selected from about 200 mg and about 400 mg Q3W. In one embodiment, the dose of pembrolizumab is about 50 mg Q3W. In one embodiment, the dose of pembrolizumab is about 100 mg Q3W. In one embodiment, the dose of pembrolizumab is about 150 mg Q3W. In one embodiment, the dose of pembrolizumab is about 200 mg Q3W. In one embodiment, the dose of pembrolizumab is about 250 mg Q3W. In one embodiment, the dose of pembrolizumab is about 300 mg Q3W. In one embodiment, the dose of pembrolizumab is about 350 mg Q3W. In one embodiment, the dose of pembrolizumab is about 400 mg Q3W. In one embodiment, the dose of pembrolizumab is about 450 mg Q3W. In one embodiment, the dose of pembrolizumab is about 500 mg Q3W.

In one embodiment, the dose of pembrolizumab is between about 50 mg and about 500 mg Q6W. In one embodiment, the dose of pembrolizumab is between about 50 mg and about 400 mg Q6W. In one embodiment, the dose of pembrolizumab is between about 50 mg and about 300 mg Q6W. In one embodiment, the dose of pembrolizumab is between about 50 mg and about 200 mg Q6W. In one embodiment, the dose of pembrolizumab is between about 50 mg and about 100 mg Q6W. In one embodiment, the dose of pembrolizumab is between about 100 mg and about 500 mg Q6W. In one embodiment, the dose of pembrolizumab is between about 100 mg and about 400 mg Q6W. In one embodiment, the dose of pembrolizumab is between about 100 mg and about 300 mg Q6W. In one embodiment, the dose of pembrolizumab is between about 100 mg and about 200 mg Q6W. In one embodiment, the dose of pembrolizumab is between about 200 mg and about 500 mg Q6W. In one embodiment, the dose of pembrolizumab is between about 200 mg and about 400 mg Q6W. In one embodiment, the dose of pembrolizumab is between about 200 mg and about 300 mg Q6W.

In one embodiment, the dose of pembrolizumab is selected from about 50 mg, about 100 mg, about 150 mg, about 200 mg, about 250 mg, about 300 mg, about 350 mg, about 400 mg, about 450 mg and about 500 mg Q6W. In one embodiment, the dose of pembrolizumab is selected from about 50 mg, about 100 mg, about 150 mg, about 200 mg, about 250 mg, about 300 mg, about 350 mg and about 400 mg Q6W. In one embodiment, the dose of pembrolizumab is selected from about 200 mg and about 400 mg Q6W. In one embodiment, the dose of pembrolizumab is about 50 mg Q6W. In one embodiment, the dose of pembrolizumab is about 100 mg Q6W. In one embodiment, the dose of pembrolizumab is about 150 mg Q6W. In one embodiment, the dose of pembrolizumab is about 200 mg Q6W. In one embodiment, the dose of pembrolizumab is about 250 mg Q6W. In one embodiment, the dose of pembrolizumab is about 300 mg Q6W. In one embodiment, the dose of pembrolizumab is about 350 mg Q6W. In one embodiment, the dose of pembrolizumab is about 400 mg Q6W. In one embodiment, the dose of pembrolizumab is about 450 mg Q6W. In one embodiment, the dose of pembrolizumab is about 500 mg Q6W.

In one embodiment, pembrolizumab is administered at a dose of about 200 mg every three weeks (Q3W) or about 400 mg every six weeks (Q6W). In one embodiment, pembrolizumab is administered at a dose of about 200 mg every three weeks (Q3W). In one embodiment, pembrolizumab is administered at a dose of about 400 mg every six weeks (Q6W).

### Combination

In an aspect, provided herein is a combination therapy comprising a dose of vorasidenib and a dose of pembrolizumab. The combination therapy is useful for the treatment of a brain tumor.

In one embodiment, the dose of vorasidenib is between about 1 mg/day and about 500 mg/day and the dose of pembrolizumab is administered at doses between about 1 mg and about 500 mg at an interval between one and ten weeks.

In one embodiment, the dose of vorasidenib is between about 35 mg/day and about 45 mg/day and the dose of pembrolizumab is between about 175 mg Q3W and about 225 mg Q3W. In one embodiment, the dose of vorasidenib is between about 30 mg/day and about 50 mg/day and the dose of pembrolizumab is between about 150 mg Q3W and about 250 mg Q3W. In one embodiment, the dose of vorasidenib is between about 25 mg/day and about 55 mg/day and the dose of pembrolizumab is between about 125 mg Q3W and about 275 mg Q3W. In one embodiment, the dose of vorasidenib is between about 20 mg/day and about 60 mg/day and the dose of pembrolizumab is between about 100 mg Q3W and about 300 mg Q3W. In one embodiment, the dose of vorasidenib is between about 15 mg/day and about 65 mg/day and the dose of pembrolizumab is between about 75 mg Q3W and about 325 mg Q3W. In one embodiment, the dose of vorasidenib is between about 10 mg/day and about 70 mg/day and the dose of pembrolizumab is between about 50 mg Q3W and about 350 mg Q3W. In one embodiment, the dose of vorasidenib is between about 5 mg/day and about 75 mg/day and the dose of pembrolizumab is between about 25 mg Q3W and about 375 mg Q3W. In one embodiment, the dose of vorasidenib is between about 1 mg/day and about 80 mg/day and the dose of pembrolizumab is between about 1 mg Q3W and about 400 mg Q3W. In one embodiment, the dose of vorasidenib is between about 1 mg/day and about 90 mg/day and the dose of pembrolizumab is between about 1 mg Q3W and about 425 mg Q3W. In one embodiment, the dose of vorasidenib is between about 1 mg/day and about 100 mg/day and the dose of pembrolizumab is between about 1 mg Q3W and about 450 mg Q3W. In one embodiment, the dose of vorasidenib is between about 1 mg/day and about 150 mg/day and the dose of pembrolizumab is between about 1 mg Q3W and about 475 mg Q3W. In one embodiment, the dose of vorasidenib is between about 1 mg/day and about 200 mg/day and the dose of pembrolizumab is between about 1 mg Q3W and about 500 mg Q3W. In one embodiment, the dose of vorasidenib is between about 1 mg/day and about 250 mg/day and the dose of pembrolizumab is between about 1 mg Q3W and about 500 mg Q3W. In one embodiment, the dose of vorasidenib is between about 1 mg/day and about 300 mg/day and the dose of pembrolizumab is between about 1 mg Q3W and about 500 mg Q3W. In one embodiment, the dose of vorasidenib is between about 1 mg/day and about 350 mg/day and the dose of pembrolizumab is between about 1 mg Q3W and about 500 mg Q3W. In one embodiment, the dose of vorasidenib is between about 1 mg/day and about 400 mg/day and the dose of pembrolizumab is between about 1 mg Q3W and about 500 mg Q3W. In one embodiment, the dose of vorasidenib is between about 1 mg/day and about 450 mg/day and the dose of pembrolizumab is between about 1 mg Q3W and about 500 mg Q3W. In one embodiment, the dose of vorasidenib is between about 1 mg/day and about 500 mg/day and the dose of pembrolizumab is between about 1 mg Q3W and about 500 mg Q3W. In one embodiment, the dose of vorasidenib is between about 35 mg/day and about 45 mg/day and the dose of pembrolizumab is between about 175 mg Q6W and about 225 mg Q6W. In one embodiment, the dose of vorasidenib is between about 30 mg/day and about 50 mg/day and the dose of pembrolizumab is between about 150 mg Q6W and about 250 mg Q6W. In one embodiment, the dose of vorasidenib is between about 25 mg/day and about 55 mg/day and the dose of pembrolizumab is between about 125 mg Q6W and about 275 mg Q6W. In one embodiment, the dose of vorasidenib is between about 20 mg/day and about 60 mg/day and the dose of pembrolizumab is between about 100 mg Q6W and about 300 mg Q6W. In one embodiment, the dose of vorasidenib is between about 15 mg/day and about 65 mg/day and the dose of pembrolizumab is between about 75 mg Q6W and about 325 mg Q6W. In one embodiment, the dose of vorasidenib is between about 10 mg/day and about 70 mg/day and the dose of pembrolizumab is between about 50 mg Q6W and about 350 mg Q6W. In one embodiment, the dose of vorasidenib is between about 5 mg/day and about 75 mg/day and the dose of pembrolizumab is between about 25 mg Q6W and about 375 mg Q6W. In one embodiment, the dose of vorasidenib is between about 1 mg/day and about 80 mg/day and the dose of pembrolizumab is between about 1 mg Q6W and about 400 mg Q6W. In one embodiment, the dose of vorasidenib is between about 1 mg/day and about 90 mg/day and the dose of pembrolizumab is between about 1 mg Q6W and about 425 mg Q6W. In one embodiment, the dose of vorasidenib is between about 1 mg/day and about 100 mg/day and the dose of pembrolizumab is between about 1 mg Q6W and about 450 mg Q6W. In one embodiment, the dose of vorasidenib is between about 1 mg/day and about 150 mg/day and the dose of pembrolizumab is between about 1 mg Q6W and about 475 mg Q6W. In one embodiment, the dose of vorasidenib is between about 1 mg/day and about 200 mg/day and the dose of pembrolizumab is between about 1 mg Q6W and about 500 mg Q6W. In one embodiment, the dose of vorasidenib is between about 1 mg/day and about 250 mg/day and the dose of pembrolizumab is between about 1 mg Q6W and about 500 mg Q6W. In one embodiment, the dose of vorasidenib is between about 1 mg/day and about 300 mg/day and the dose of pembrolizumab is between about 1 mg Q6W and about 500 mg Q6W. In one embodiment, the dose of vorasidenib is between about 1 mg/day and about 350 mg/day and the dose of pembrolizumab is between about 1 mg Q6W and about 500 mg Q6W. In one embodiment, the dose of vorasidenib is between about 1 mg/day and about 400 mg/day and the dose of pembrolizumab is between about 1 mg Q6W and about 500 mg Q6W. In one embodiment, the dose of vorasidenib is between about 1 mg/day and about 450 mg/day and the dose of pembrolizumab is between about 1 mg Q6W and about 500 mg Q6W. In one embodiment, the dose of vorasidenib is between about 1 mg/day and about 500 mg/day and the dose of pembrolizumab is between about 1 mg Q6W and about 500 mg Q6W.

In one embodiment, the dose of vorasidenib is between about 35 mg and about 45 mg once daily and the dose of pembrolizumab is between about 175 mg Q3W and about 225 mg Q3W. In one embodiment, the dose of vorasidenib is between about 30 mg and about 50 mg once daily and the dose of pembrolizumab is between about 150 mg Q3W and about 250 mg Q3W. In one embodiment, the dose of vorasidenib is between about 25 mg and about 55 mg once daily and the dose of pembrolizumab is between about 125 mg Q3W and about 275 mg Q3W. In one embodiment, the dose of vorasidenib is between about 20 mg and about 60 mg once daily and the dose of pembrolizumab is between about 100 mg Q3W and about 300 mg Q3W. In one embodiment, the dose of vorasidenib is between about 15 mg and about 65 mg once daily and the dose of pembrolizumab is between about 75 mg Q3W and about 325 mg Q3W. In one embodiment, the dose of vorasidenib is between about 10 mg and about 70 mg once daily and the dose of pembrolizumab is between about 50 mg Q3W and about 350 mg Q3W. In one embodiment, the dose of vorasidenib is between about 5 mg and about 75 mg once daily and the dose of pembrolizumab is between about 25 mg Q3W and about 375 mg Q3W. In one embodiment, the dose of vorasidenib is between about 1 mg and about 80 mg once daily and the dose of pembrolizumab is between about 1 mg Q3W and about 400 mg Q3W. In one embodiment, the dose of vorasidenib is between about 1 mg and about 90 mg once daily and the dose of pembrolizumab is between about 1 mg Q3W and about 425 mg Q3W. In one embodiment, the dose of vorasidenib is between about 1 mg and about 100 mg once daily and the dose of pembrolizumab is between about 1 mg Q3W and about 450 mg Q3W. In one embodiment, the dose of vorasidenib is between about 1 mg and about 150 mg once daily and the dose of pembrolizumab is between about 1 mg Q3W and about 475 mg Q3W. In one embodiment, the dose of vorasidenib is between about 1 mg and about 200 mg once daily and the dose of pembrolizumab is between about 1 mg Q3W and about 500 mg Q3W. In one embodiment, the dose of vorasidenib is between about 1 mg and about 250 mg once daily and the dose of pembrolizumab is between about 1 mg Q3W and about 500 mg Q3W. In one embodiment, the dose of vorasidenib is between about 1 mg and about 300 mg once daily and the dose of pembrolizumab is between about 1 mg Q3W and about 500 mg Q3W. In one embodiment, the dose of vorasidenib is between about 1 mg and about 350 mg once daily and the dose of pembrolizumab is between about 1 mg Q3W and about 500 mg Q3W. In one embodiment, the dose of vorasidenib is between about 1 mg and about 400 mg once daily and the dose of pembrolizumab is between about 1 mg Q3W and about 500 mg Q3W. In one embodiment, the dose of vorasidenib is between about 1 mg and about 450 mg once daily and the dose of pembrolizumab is between about 1 mg Q3W and about 500 mg Q3W. In one embodiment, the dose of vorasidenib is between about 1 mg and about 500 mg once daily and the dose of pembrolizumab is between about 1 mg Q3W and about 500 mg Q3W.

In one embodiment, the dose of vorasidenib is between about 35 mg and about 45 mg once daily and the dose of pembrolizumab is between about 175 mg Q6W and about 225 mg Q6W. In one embodiment, the dose of vorasidenib is between about 30 mg and about 50 mg once daily and the dose of pembrolizumab is between about 150 mg Q6W and about 250 mg Q6W. In one embodiment, the dose of vorasidenib is between about 25 mg and about 55 mg once daily and the dose of pembrolizumab is between about 125 mg Q6W and about 275 mg Q6W. In one embodiment, the dose of vorasidenib is between about 20 mg and about 60 mg once daily and the dose of pembrolizumab is between about 100 mg Q6W and about 300 mg Q6W. In one embodiment, the dose of vorasidenib is between about 15 mg and about 65 mg once daily and the dose of pembrolizumab is between about 75 mg Q6W and about 325 mg Q6W. In one embodiment, the dose of vorasidenib is between about 10 mg and about 70 mg once daily and the dose of pembrolizumab is between about 50 mg Q6W and about 350 mg Q6W. In one embodiment, the dose of vorasidenib is between about 5 mg and about 75 mg once daily and the dose of pembrolizumab is between about 25 mg Q6W and about 375 mg Q6W. In one embodiment, the dose of vorasidenib is between about 1 mg and about 80 mg once daily and the dose of pembrolizumab is between about 1 mg Q6W and about 400 mg Q6W. In one embodiment, the dose of vorasidenib is between about 1 mg and about 90 mg once daily and the dose of pembrolizumab is between about 1 mg Q6W and about 425 mg Q6W. In one embodiment, the dose of vorasidenib is between about 1 mg and about 100 mg once daily and the dose of pembrolizumab is between about 1 mg Q6W and about 450 mg Q6W. In one embodiment, the dose of vorasidenib is between about 1 mg and about 150 mg once daily and the dose of pembrolizumab is between about 1 mg Q6W and about 475 mg Q6W. In one embodiment, the dose of vorasidenib is between about 1 mg and about 200 mg once daily and the dose of pembrolizumab is between about 1 mg Q6W and about 500 mg Q6W. In one embodiment, the dose of vorasidenib is between about 1 mg and about 250 mg once daily and the dose of pembrolizumab is between about 1 mg Q6W and about 500 mg Q6W. In one embodiment, the dose of vorasidenib is between about 1 mg and about 300 mg once daily and the dose of pembrolizumab is between about 1 mg Q6W and about 500 mg Q6W. In one embodiment, the dose of vorasidenib is between about 1 mg and about 350 mg once daily and the dose of pembrolizumab is between about 1 mg Q6W and about 500 mg Q6W. In one embodiment, the dose of vorasidenib is between about 1 mg and about 400 mg once daily and the dose of pembrolizumab is between about 1 mg Q6W and about 500 mg Q6W. In one embodiment, the dose of vorasidenib is between about 1 mg and about 450 mg once daily and the dose of pembrolizumab is between about 1 mg Q6W and about 500 mg Q6W. In one embodiment, the dose of vorasidenib is between about 1 mg and about 500 mg once daily and the dose of pembrolizumab is between about 1 mg Q6W and about 500 mg Q6W.

In one embodiment, the dose of vorasidenib is between about 35 mg and about 45 mg twice daily and the dose of pembrolizumab is between about 175 mg Q3W and about 225 mg Q3W. In one embodiment, the dose of vorasidenib is between about 30 mg and about 50 mg twice daily and the dose of pembrolizumab is between about 150 mg Q3W and about 250 mg Q3W. In one embodiment, the dose of vorasidenib is between about 25 mg and about 55 mg twice daily and the dose of pembrolizumab is between about 125 mg Q3W and about 275 mg Q3W. In one embodiment, the dose of vorasidenib is between about 20 mg and about 60 mg twice daily and the dose of pembrolizumab is between about 100 mg Q3W and about 300 mg Q3W. In one embodiment, the dose of vorasidenib is between about 15 mg and about 65 mg twice daily and the dose of pembrolizumab is between about 75 mg Q3W and about 325 mg Q3W. In one embodiment, the dose of vorasidenib is between about 10 mg and about 70 mg twice daily and the dose of pembrolizumab is between about 50 mg Q3W and about 350 mg Q3W. In one embodiment, the dose of vorasidenib is between about 5 mg and about 75 mg twice daily and the dose of pembrolizumab is between about 25 mg Q3W and about 375 mg Q3W. In one embodiment, the dose of vorasidenib is between about 1 mg and about 80 mg twice daily and the dose of pembrolizumab is between about 1 mg Q3W and about 400 mg Q3W. In one embodiment, the dose of vorasidenib is between about 1 mg and about 90 mg twice daily and the dose of pembrolizumab is between about 1 mg Q3W and about 425 mg Q3W. In one embodiment, the dose of vorasidenib is between about 1 mg and about 100 mg twice daily and the dose of pembrolizumab is between about 1 mg Q3W and about 450 mg Q3W. In one embodiment, the dose of vorasidenib is between about 1 mg and about 150 mg twice daily and the dose of pembrolizumab is between about 1 mg Q3W and about 475 mg Q3W. In one embodiment, the dose of vorasidenib is between about 1 mg and about 200 mg twice daily and the dose of pembrolizumab is between about 1 mg Q3W and about 500 mg Q3W. In one embodiment, the dose of vorasidenib is between about 1 mg and about 250 mg twice daily and the dose of pembrolizumab is between about 1 mg Q3W and about 500 mg Q3W. In one embodiment, the dose of vorasidenib is between about 1 mg and about 300 mg twice daily and the dose of pembrolizumab is between about 1 mg Q3W and about 500 mg Q3W. In one embodiment, the dose of vorasidenib is between about 1 mg and about 350 mg twice daily and the dose of pembrolizumab is between about 1 mg Q3W and about 500 mg Q3W. In one embodiment, the dose of vorasidenib is between about 1 mg and about 400 mg twice daily and the dose of pembrolizumab is between about 1 mg Q3W and about 500 mg Q3W. In one embodiment, the dose of vorasidenib is between about 1 mg and about 450 mg twice daily and the dose of pembrolizumab is between about 1 mg Q3W and about 500 mg Q3W. In one embodiment, the dose of vorasidenib is between about 1 mg and about 500 mg twice daily and the dose of pembrolizumab is between about 1 mg Q3W and about 500 mg Q3W.

In one embodiment, the dose of vorasidenib is between about 35 mg and about 45 mg twice daily and the dose of pembrolizumab is between about 175 mg Q6W and about 225 mg Q6W. In one embodiment, the dose of vorasidenib is between about 30 mg and about 50 mg twice daily and the dose of pembrolizumab is between about 150 mg Q6W and about 250 mg Q6W. In one embodiment, the dose of vorasidenib is between about 25 mg and about 55 mg twice daily and the dose of pembrolizumab is between about 125 mg Q6W and about 275 mg Q6W. In one embodiment, the dose of vorasidenib is between about 20 mg and about 60 mg twice daily and the dose of pembrolizumab is between about 100 mg Q6W and about 300 mg Q6W. In one embodiment, the dose of vorasidenib is between about 15 mg and about 65 mg twice daily and the dose of pembrolizumab is between about 75 mg Q6W and about 325 mg Q6W. In one embodiment, the dose of vorasidenib is between about 10 mg and about 70 mg twice daily and the dose of pembrolizumab is between about 50 mg Q6W and about 350 mg Q6W. In one embodiment, the dose of vorasidenib is between about 5 mg and about 75 mg twice daily and the dose of pembrolizumab is between about 25 mg Q6W and about 375 mg Q6W. In one embodiment, the dose of vorasidenib is between about 1 mg and about 80 mg twice daily and the dose of pembrolizumab is between about 1 mg Q6W and about 400 mg Q6W. In one embodiment, the dose of vorasidenib is between about 1 mg and about 90 mg twice daily and the dose of pembrolizumab is between about 1 mg Q6W and about 425 mg Q6W. In one embodiment, the dose of vorasidenib is between about 1 mg and about 100 mg twice daily and the dose of pembrolizumab is between about 1 mg Q6W and about 450 mg Q6W. In one embodiment, the dose of vorasidenib is between about 1 mg and about 150 mg twice daily and the dose of pembrolizumab is between about 1 mg Q6W and about 475 mg Q6W. In one embodiment, the dose of vorasidenib is between about 1 mg and about 200 mg twice daily and the dose of pembrolizumab is between about 1 mg Q6W and about 500 mg Q6W. In one embodiment, the dose of vorasidenib is between about 1 mg and about 250 mg twice daily and the dose of pembrolizumab is between about 1 mg Q6W and about 500 mg Q6W. In one embodiment, the dose of vorasidenib is between about 1 mg and about 300 mg twice daily and the dose of pembrolizumab is between about 1 mg Q6W and about 500 mg Q6W. In one embodiment, the dose of vorasidenib is between about 1 mg and about 350 mg twice daily and the dose of pembrolizumab is between about 1 mg Q6W and about 500 mg Q6W. In one embodiment, the dose of vorasidenib is between about 1 mg and about 400 mg twice daily and the dose of pembrolizumab is between about 1 mg Q6W and about 500 mg Q6W. In one embodiment, the dose of vorasidenib is between about 1 mg and about 450 mg twice daily and the dose of pembrolizumab is between about 1 mg Q6W and about 500 mg Q6W. In one embodiment, the dose of vorasidenib is between about 1 mg and about 500 mg twice daily and the dose of pembrolizumab is between about 1 mg Q6W and about 500 mg Q6W.

In one embodiment, the dose comprises about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 250 mg/day, about 300 mg/day, about 350 mg/day, about 400 mg/day, about 450 mg/day or about 500 mg/day vorasidenib and about 1 mg Q3W, about 2 mg Q3W, about 3 mg Q3W, about 4 mg Q3W, about 5 mg Q3W, about 6 mg Q3W, about 7 mg Q3W, about 8 mg Q3W, about 9 mg Q3W, about 10 mg Q3W, about 15 mg Q3W, about 20 mg Q3W, about 25 mg Q3W, about 30 mg Q3W, about 35 mg Q3W, about 40 mg Q3W, about 45 mg Q3W, about 50 mg Q3W, about 55 mg Q3W, about 60 mg Q3W, about 65 mg Q3W, about 70 mg Q3W, about 75 mg Q3W, about 80 mg Q3W, about 85 mg Q3W, about 90 mg Q3W, about 95 mg Q3W, about 100 mg Q3W, about 110 mg Q3W, about 120 mg Q3W, about 130 mg Q3W, about 140 mg Q3W, about 150 mg Q3W, about 160 mg Q3W, about 170 mg Q3W, about 180 mg Q3W, about 190 mg Q3W, about 200 mg Q3W, about 250 mg Q3W, about 300 mg Q3W, about 350 mg Q3W, about 400 mg Q3W, about 450 mg Q3W or about 500 mg Q3W pembrolizumab.

In one embodiment, the dose comprises about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day, about 200 mg/day, about 250 mg/day, about 300 mg/day, about 350 mg/day, about 400 mg/day, about 450 mg/day or about 500 mg/day vorasidenib and about 1 mg Q6W, about 2 mg Q6W, about 3 mg Q6W, about 4 mg Q6W, about 5 mg Q6W, about 6 mg Q6W, about 7 mg Q6W, about 8 mg Q6W, about 9 mg Q6W, about 10 mg Q6W, about 15 mg Q6W, about 20 mg Q6W, about 25 mg Q6W, about 30 mg Q6W, about 35 mg Q6W, about 40 mg Q6W, about 45 mg Q6W, about 50 mg Q6W, about 55 mg Q6W, about 60 mg Q6W, about 65 mg Q6W, about 70 mg Q6W, about 75 mg Q6W, about 80 mg Q6W, about 85 mg Q6W, about 90 mg Q6W, about 95 mg Q6W, about 100 mg Q6W, about 110 mg Q6W, about 120 mg Q6W, about 130 mg Q6W, about 140 mg Q6W, about 150 mg Q6W, about 160 mg Q6W, about 170 mg Q6W, about 180 mg Q6W, about 190 mg Q6W, about 200 mg Q6W, about 250 mg Q6W, about 300 mg Q6W, about 350 mg Q6W, about 400 mg Q6W, about 450 mg Q6W or about 500 mg Q6W pembrolizumab.

In one embodiment, the dose comprises about 1 mg once daily, about 2 mg once daily, about 3 mg once daily, about 4 mg once daily, about 5 mg once daily, about 6 mg once daily, about 7 mg once daily, about 8 mg once daily, about 9 mg once daily, about 10 mg once daily, about 15 mg once daily, about 20 mg once daily, about 25 mg once daily, about 30 mg once daily, about 35 mg once daily, about 40 mg once daily, about 45 mg once daily, about 50 mg once daily, about 55 mg once daily, about 60 mg once daily, about 65 mg once daily, about 70 mg once daily, about 75 mg once daily, about 80 mg once daily, about 85 mg once daily, about 90 mg once daily, about 95 mg once daily, about 100 mg once daily, about 110 mg once daily, about 120 mg once daily, about 130 mg once daily, about 140 mg once daily, about 150 mg once daily, about 160 mg once daily, about 170 mg once daily, about 180 mg once daily, about 190 mg once daily, about 200 mg once daily, about 250 mg once daily, about 300 mg once daily, about 350 mg once daily, about 400 mg once daily, about 450 mg once daily or about 500 mg once daily vorasidenib and about 1 mg Q3W, about 2 mg Q3W, about 3 mg Q3W, about 4 mg Q3W, about 5 mg Q3W, about 6 mg Q3W, about 7 mg Q3W, about 8 mg Q3W, about 9 mg Q3W, about 10 mg Q3W, about 15 mg Q3W, about 20 mg Q3W, about 25 mg Q3W, about 30 mg Q3W, about 35 mg Q3W, about 40 mg Q3W, about 45 mg Q3W, about 50 mg Q3W, about 55 mg Q3W, about 60 mg Q3W, about 65 mg Q3W, about 70 mg Q3W, about 75 mg Q3W, about 80 mg Q3W, about 85 mg Q3W, about 90 mg Q3W, about 95 mg Q3W, about 100 mg Q3W, about 110 mg Q3W, about 120 mg Q3W, about 130 mg Q3W, about 140 mg Q3W, about 150 mg Q3W, about 160 mg Q3W, about 170 mg Q3W, about 180 mg Q3W, about 190 mg Q3W, about 200 mg Q3W, about 250 mg Q3W, about 300 mg Q3W, about 350 mg Q3W, about 400 mg Q3W, about 450 mg Q3W or about 500 mg Q3W pembrolizumab.

In one embodiment, the dose comprises about 1 mg once daily, about 2 mg once daily, about 3 mg once daily, about 4 mg once daily, about 5 mg once daily, about 6 mg once daily, about 7 mg once daily, about 8 mg once daily, about 9 mg once daily, about 10 mg once daily, about 15 mg once daily, about 20 mg once daily, about 25 mg once daily, about 30 mg once daily, about 35 mg once daily, about 40 mg once daily, about 45 mg once daily, about 50 mg once daily, about 55 mg once daily, about 60 mg once daily, about 65 mg once daily, about 70 mg once daily, about 75 mg once daily, about 80 mg once daily, about 85 mg once daily, about 90 mg once daily, about 95 mg once daily, about 100 mg once daily, about 110 mg once daily, about 120 mg once daily, about 130 mg once daily, about 140 mg once daily, about 150 mg once daily, about 160 mg once daily, about 170 mg once daily, about 180 mg once daily, about 190 mg once daily, about 200 mg once daily, about 250 mg once daily, about 300 mg once daily, about 350 mg once daily, about 400 mg once daily, about 450 mg once daily or about 500 mg once daily vorasidenib and about 1 mg Q6W, about 2 mg Q6W, about 3 mg Q6W, about 4 mg Q6W, about 5 mg Q6W, about 6 mg Q6W, about 7 mg Q6W, about 8 mg Q6W, about 9 mg Q6W, about 10 mg Q6W, about 15 mg Q6W, about 20 mg Q6W, about 25 mg Q6W, about 30 mg Q6W, about 35 mg Q6W, about 40 mg Q6W, about 45 mg Q6W, about 50 mg Q6W, about 55 mg Q6W, about 60 mg Q6W, about 65 mg Q6W, about 70 mg Q6W, about 75 mg Q6W, about 80 mg Q6W, about 85 mg Q6W, about 90 mg Q6W, about 95 mg Q6W, about 100 mg Q6W, about 110 mg Q6W, about 120 mg Q6W, about 130 mg Q6W, about 140 mg Q6W, about 150 mg Q6W, about 160 mg Q6W, about 170 mg Q6W, about 180 mg Q6W, about 190 mg Q6W, about 200 mg Q6W, about 250 mg Q6W, about 300 mg Q6W, about 350 mg Q6W, about 400 mg Q6W, about 450 mg Q6W or about 500 mg Q6W pembrolizumab.

In one embodiment, the dose comprises about 1 mg twice daily, about 2 mg twice daily, about 3 mg twice daily, about 4 mg twice daily, about 5 mg twice daily, about 6 mg twice daily, about 7 mg twice daily, about 8 mg twice daily, about 9 mg twice daily, about 10 mg twice daily, about 15 mg twice daily, about 20 mg twice daily, about 25 mg twice daily, about 30 mg twice daily, about 35 mg twice daily, about 40 mg twice daily, about 45 mg twice daily, about 50 mg twice daily, about 55 mg twice daily, about 60 mg twice daily, about 65 mg twice daily, about 70 mg twice daily, about 75 mg twice daily, about 80 mg twice daily, about 85 mg twice daily, about 90 mg twice daily, about 95 mg twice daily, about 100 mg twice daily, about 110 mg twice daily, about 120 mg twice daily, about 130 mg twice daily, about 140 mg twice daily, about 150 mg twice daily, about 160 mg twice daily, about 170 mg twice daily, about 180 mg twice daily, about 190 mg twice daily, about 200 mg twice daily, about 250 mg twice daily, about 300 mg twice daily, about 350 mg twice daily, about 400 mg twice daily, about 450 mg twice daily or about 500 mg twice daily vorasidenib and about 1 mg Q3W, about 2 mg Q3W, about 3 mg Q3W, about 4 mg Q3W, about 5 mg Q3W, about 6 mg Q3W, about 7 mg Q3W, about 8 mg Q3W, about 9 mg Q3W, about 10 mg Q3W, about 15 mg Q3W, about 20 mg Q3W, about 25 mg Q3W, about 30 mg Q3W, about 35 mg Q3W, about 40 mg Q3W, about 45 mg Q3W, about 50 mg Q3W, about 55 mg Q3W, about 60 mg Q3W, about 65 mg Q3W, about 70 mg Q3W, about 75 mg Q3W, about 80 mg Q3W, about 85 mg Q3W, about 90 mg Q3W, about 95 mg Q3W, about 100 mg Q3W, about 110 mg Q3W, about 120 mg Q3W, about 130 mg Q3W, about 140 mg Q3W, about 150 mg Q3W, about 160 mg Q3W, about 170 mg Q3W, about 180 mg Q3W, about 190 mg Q3W, about 200 mg Q3W, about 250 mg Q3W, about 300 mg Q3W, about 350 mg Q3W, about 400 mg Q3W, about 450 mg Q3W or about 500 mg Q3W pembrolizumab.

In one embodiment, the dose comprises about 1 mg twice daily, about 2 mg twice daily, about 3 mg twice daily, about 4 mg twice daily, about 5 mg twice daily, about 6 mg twice daily, about 7 mg twice daily, about 8 mg twice daily, about 9 mg twice daily, about 10 mg twice daily, about 15 mg twice daily, about 20 mg twice daily, about 25 mg twice daily, about 30 mg twice daily, about 35 mg twice daily, about 40 mg twice daily, about 45 mg twice daily, about 50 mg twice daily, about 55 mg twice daily, about 60 mg twice daily, about 65 mg twice daily, about 70 mg twice daily, about 75 mg twice daily, about 80 mg twice daily, about 85 mg twice daily, about 90 mg twice daily, about 95 mg twice daily, about 100 mg twice daily, about 110 mg twice daily, about 120 mg twice daily, about 130 mg twice daily, about 140 mg twice daily, about 150 mg twice daily, about 160 mg twice daily, about 170 mg twice daily, about 180 mg twice daily, about 190 mg twice daily, about 200 mg twice daily, about 250 mg twice daily, about 300 mg twice daily, about 350 mg twice daily, about 400 mg twice daily, about 450 mg twice daily or about 500 mg twice daily vorasidenib and about 1 mg Q6W, about 2 mg Q6W, about 3 mg Q6W, about 4 mg Q6W, about 5 mg Q6W, about 6 mg Q6W, about 7 mg Q6W, about 8 mg Q6W, about 9 mg Q6W, about 10 mg Q6W, about 15 mg Q6W, about 20 mg Q6W, about 25 mg Q6W, about 30 mg Q6W, about 35 mg Q6W, about 40 mg Q6W, about 45 mg Q6W, about 50 mg Q6W, about 55 mg Q6W, about 60 mg Q6W, about 65 mg Q6W, about 70 mg Q6W, about 75 mg Q6W, about 80 mg Q6W, about 85 mg Q6W, about 90 mg Q6W, about 95 mg Q6W, about 100 mg Q6W, about 110 mg Q6W, about 120 mg Q6W, about 130 mg Q6W, about 140 mg Q6W, about 150 mg Q6W, about 160 mg Q6W, about 170 mg Q6W, about 180 mg Q6W, about 190 mg Q6W, about 200 mg Q6W, about 250 mg Q6W, about 300 mg Q6W, about 350 mg Q6W, about 400 mg Q6W, about 450 mg Q6W or about 500 mg Q6W pembrolizumab.

In one embodiment, the dose comprises about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day or about 100 mg/day vorasidenib and about 10 mg Q3W, about 15 mg Q3W, about 20 mg Q3W, about 25 mg Q3W, about 30 mg Q3W, about 35 mg Q3W, about 40 mg Q3W, about 45 mg Q3W, about 50 mg Q3W, about 55 mg Q3W, about 60 mg Q3W, about 65 mg Q3W, about 70 mg Q3W, about 75 mg Q3W, about 80 mg Q3W, about 85 mg Q3W, about 90 mg Q3W, about 95 mg Q3W. about 100 mg Q3W, about 150 mg Q3W, or about 200 mg Q3W pembrolizumab.

In one embodiment, the dose comprises about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day or about 100 mg/day vorasidenib and about 10 mg Q6W, about 15 mg Q6W, about 20 mg Q6W, about 25 mg Q6W, about 30 mg Q6W, about 35 mg Q6W, about 40 mg Q6W, about 45 mg Q6W, about 50 mg Q6W, about 55 mg Q6W, about 60 mg Q6W, about 65 mg Q6W, about 70 mg Q6W, about 75 mg Q6W, about 80 mg Q6W, about 85 mg Q6W, about 90 mg Q6W, about 95 mg Q6W. about 100 mg Q6W, about 150 mg Q6W, or about 200 mg Q6W pembrolizumab.

In one embodiment, the dose comprises about 10 mg once daily, about 15 mg once daily, about 20 mg once daily, about 25 mg once daily, about 30 mg once daily, about 35 mg once daily, about 40 mg once daily, about 45 mg once daily, about 50 mg once daily, about 55 mg once daily, about 60 mg once daily, about 65 mg once daily, about 70 mg once daily, about 75 mg once daily, about 80 mg once daily, about 85 mg once daily, about 90 mg once daily, about 95 mg once daily or about 100 mg once daily vorasidenib and about 10 mg Q3W, about 15 mg Q3W, about 20 mg Q3W, about 25 mg Q3W, about 30 mg Q3W, about 35 mg Q3W, about 40 mg Q3W, about 45 mg Q3W, about 50 mg Q3W, about 55 mg Q3W, about 60 mg Q3W, about 65 mg Q3W, about 70 mg Q3W, about 75 mg Q3W, about 80 mg Q3W, about 85 mg Q3W, about 90 mg Q3W, about 95 mg Q3W. about 100 mg Q3W, about 150 mg Q3W, or about 200 mg Q3W pembrolizumab.

In one embodiment, the dose comprises about 10 mg once daily, about 15 mg once daily, about 20 mg once daily, about 25 mg once daily, about 30 mg once daily, about 35 mg once daily, about 40 mg once daily, about 45 mg once daily, about 50 mg once daily, about 55 mg once daily, about 60 mg once daily, about 65 mg once daily, about 70 mg once daily, about 75 mg once daily, about 80 mg once daily, about 85 mg once daily, about 90 mg once daily, about 95 mg once daily or about 100 mg once daily vorasidenib and about 10 mg Q6W, about 15 mg Q6W, about 20 mg Q6W, about 25 mg Q6W, about 30 mg Q6W, about 35 mg Q6W, about 40 mg Q6W, about 45 mg Q6W, about 50 mg Q6W, about 55 mg Q6W, about 60 mg Q6W, about 65 mg Q6W, about 70 mg Q6W, about 75 mg Q6W, about 80 mg Q6W, about 85 mg Q6W, about 90 mg Q6W, about 95 mg Q6W. about 100 mg Q6W, about 150 mg Q6W, or about 200 mg Q6W pembrolizumab.

In one embodiment, the dose comprises about 10 mg twice daily, about 15 mg twice daily, about 20 mg twice daily, about 25 mg twice daily, about 30 mg twice daily, about 35 mg twice daily, about 40 mg twice daily, about 45 mg twice daily, about 50 mg twice daily, about 55 mg twice daily, about 60 mg twice daily, about 65 mg twice daily, about 70 mg twice daily, about 75 mg twice daily, about 80 mg twice daily, about 85 mg twice daily, about 90 mg twice daily, about 95 mg twice daily or about 100 mg twice daily vorasidenib and about 10 mg Q3W, about 15 mg Q3W, about 20 mg Q3W, about 25 mg Q3W, about 30 mg Q3W, about 35 mg Q3W, about 40 mg Q3W, about 45 mg Q3W, about 50 mg Q3W, about 55 mg Q3W, about 60 mg Q3W, about 65 mg Q3W, about 70 mg Q3W, about 75 mg Q3W, about 80 mg Q3W, about 85 mg Q3W, about 90 mg Q3W, about 95 mg Q3W. about 100 mg Q3W, about 150 mg Q3W, or about 200 mg Q3W pembrolizumab.

In one embodiment, the dose comprises about 10 mg twice daily, about 15 mg twice daily, about 20 mg twice daily, about 25 mg twice daily, about 30 mg twice daily, about 35 mg twice daily, about 40 mg twice daily, about 45 mg twice daily, about 50 mg twice daily, about 55 mg twice daily, about 60 mg twice daily, about 65 mg twice daily, about 70 mg twice daily, about 75 mg twice daily, about 80 mg twice daily, about 85 mg twice daily, about 90 mg twice daily, about 95 mg twice daily or about 100 mg twice daily vorasidenib and about 10 mg Q6W, about 15 mg Q6W, about 20 mg Q6W, about 25 mg Q6W, about 30 mg Q6W, about 35 mg Q6W, about 40 mg Q6W, about 45 mg Q6W, about 50 mg Q6W, about 55 mg Q6W, about 60 mg Q6W, about 65 mg Q6W, about 70 mg Q6W, about 75 mg Q6W, about 80 mg Q6W, about 85 mg Q6W, about 90 mg Q6W, about 95 mg Q6W. about 100 mg Q6W, about 150 mg Q6W, or about 200 mg Q6W pembrolizumab.

In one embodiment, the dose comprises about 40 mg/day vorasidenib and 200 mg Q3W pembrolizumab. In one embodiment, the dose comprises about 40 mg once daily vorasidenib and 200 mg Q3W pembrolizumab. In one embodiment, the dose comprises about 40 mg twice daily vorasidenib and 200 mg Q3W pembrolizumab. In one embodiment, the dose comprises about 40 mg/day vorasidenib and 200 mg Q6W pembrolizumab. In one embodiment, the dose comprises about 40 mg once daily vorasidenib and 200 mg Q6W pembrolizumab. In one embodiment, the dose comprises about 40 mg twice daily vorasidenib and 200 mg Q6W pembrolizumab.

In one embodiment, the dose comprises about 20 mg/day vorasidenib and 200 mg Q3W pembrolizumab. In one embodiment, the dose comprises about 20 mg once daily vorasidenib and 200 mg Q3W pembrolizumab. In one embodiment, the dose comprises about 20 mg twice daily vorasidenib and 200 mg Q3W pembrolizumab. In one embodiment, the dose comprises about 20 mg/day vorasidenib and 200 mg Q6W pembrolizumab. In one embodiment, the dose comprises about 20 mg once daily vorasidenib and 200 mg Q6W pembrolizumab. In one embodiment, the dose comprises about 20 mg twice daily vorasidenib and 200 mg Q6W pembrolizumab.

In one embodiment, vorasidenib is administered at a dose between about 10 mg/day and about 100 mg/day and pembrolizumab is administered at a dose between about 10 mg and about 500 mg every three weeks (Q3W) or every 6 weeks (Q6W). In one embodiment, vorasidenib is administered at a dose between about 10 mg/day and about 100 mg/day and pembrolizumab is administered at a dose between about 100 mg and about 400 mg Q3W or Q6W. In one embodiment, vorasidenib is administered at a dose between about 10 mg/day and about 100 mg/day and pembrolizumab is administered at a dose between about 200 mg and about 400 mg Q3W or Q6W. In one embodiment, vorasidenib is administered at a dose between about 10 mg/day and about 100 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg/day and about 100 mg/day and pembrolizumab is administered at a dose of about 100 mg, about 200 mg or about 400 mg Q3W or Q6W. In one embodiment, vorasidenib is administered at a dose between about 10 mg/day and about 100 mg/day and pembrolizumab is administered at a dose of about 200 mg or about 400 mg Q3W or Q6W. In one embodiment, vorasidenib is administered at a dose between about 10 mg/day and about 100 mg/day and pembrolizumab is administered at a dose between about 100 mg and about 300 mg Q3W. In one embodiment, vorasidenib is administered at a dose between about 10 mg/day and about 100 mg/day and pembrolizumab is administered at a dose of about 100 mg, 200 mg or 300 mg Q3W. In one embodiment, vorasidenib is administered at a dose between about 10 mg/day and about 100 mg/day and pembrolizumab is administered at a dose of about 200 mg Q3W. In one embodiment, vorasidenib is administered at a dose between about 10 mg/day and about 100 mg/day and pembrolizumab is administered at a dose between about 200 mg and about 500 mg Q3W. In one embodiment, vorasidenib is administered at a dose between about 10 mg/day and about 100 mg/day and pembrolizumab is administered at a dose of about 200 mg, 400 mg or 500 mg Q3W. In one embodiment, vorasidenib is administered at a dose between about 10 mg/day and about 100 mg/day and pembrolizumab is administered at a dose of about 400 mg Q3W.

In one embodiment, vorasidenib is administered at a dose between about 20 mg/day and about 40 mg/day and pembrolizumab is administered at a dose between about 10 mg and about 500 mg every three weeks (Q3W) or every 6 weeks (Q6W). In one embodiment, vorasidenib is administered at a dose between about 20 mg/day and about 40 mg/day and pembrolizumab is administered at a dose between about 100 mg and about 400 mg Q3W or Q6W. In one embodiment, vorasidenib is administered at a dose between about 20 mg/day and about 40 mg/day and pembrolizumab is administered at a dose between about 200 mg and about 400 mg Q3W or Q6W. In one embodiment, vorasidenib is administered at a dose between about 20 mg/day and about 40 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 20 mg/day and about 40 mg/day and pembrolizumab is administered at a dose of about 100 mg, about 200 mg or about 400 mg Q3W or Q6W. In one embodiment, vorasidenib is administered at a dose between about 20 mg/day and about 40 mg/day and pembrolizumab is administered at a dose of about 200 mg or about 400 mg Q3W or Q6W. In one embodiment, vorasidenib is administered at a dose between about 20 mg/day and about 40 mg/day and pembrolizumab is administered at a dose between about 100 mg and about 300 mg Q3W. In one embodiment, vorasidenib is administered at a dose between about 20 mg/day and about 40 mg/day and pembrolizumab is administered at a dose of about 100 mg, 200 mg or 300 mg Q3W. In one embodiment, vorasidenib is administered at a dose between about 20 mg/day and about 40 mg/day and pembrolizumab is administered at a dose of about 200 mg Q3W. In one embodiment, vorasidenib is administered at a dose between about 20 mg/day and about 40 mg/day and pembrolizumab is administered at a dose between about 200 mg and about 500 mg Q3W. In one embodiment, vorasidenib is administered at a dose between about 20 mg/day and about 40 mg/day and pembrolizumab is administered at a dose of about 200 mg, 400 mg or 500 mg Q3W. In one embodiment, vorasidenib is administered at a dose between about 20 mg/day and about 40 mg/day and pembrolizumab is administered at a dose of about 400 mg Q3W.

In one embodiment, vorasidenib is administered at a dose between about 10 mg/day and about 50 mg/day and pembrolizumab is administered at a dose between about 10 mg and about 500 mg every three weeks (Q3W) or every 6 weeks (Q6W). In one embodiment, vorasidenib is administered at a dose between about 10 mg/day and about 50 mg/day and pembrolizumab is administered at a dose between about 100 mg and about 400 mg Q3W or Q6W. In one embodiment, vorasidenib is administered at a dose between about 10 mg/day and about 50 mg/day and pembrolizumab is administered at a dose between about 200 mg and about 400 mg Q3W or Q6W. In one embodiment, vorasidenib is administered at a dose between about 10 mg/day and about 50 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg/day and about 50 mg/day and pembrolizumab is administered at a dose of about 100 mg, about 200 mg or about 400 mg Q3W or Q6W. In one embodiment, vorasidenib is administered at a dose between about 10 mg/day and about 50 mg/day and pembrolizumab is administered at a dose of about 200 mg or about 400 mg Q3W or Q6W. In one embodiment, vorasidenib is administered at a dose between about 10 mg/day and about 50 mg/day and pembrolizumab is administered at a dose between about 100 mg and about 300 mg Q3W. In one embodiment, vorasidenib is administered at a dose between about 10 mg/day and about 50 mg/day and pembrolizumab is administered at a dose of about 100 mg, 200 mg or 300 mg Q3W. In one embodiment, vorasidenib is administered at a dose between about 10 mg/day and about 50 mg/day and pembrolizumab is administered at a dose of about 200 mg Q3W. In one embodiment, vorasidenib is administered at a dose between about 10 mg/day and about 50 mg/day and pembrolizumab is administered at a dose between about 200 mg and about 500 mg Q3W. In one embodiment, vorasidenib is administered at a dose between about 10 mg/day and about 50 mg/day and pembrolizumab is administered at a dose of about 200 mg, 400 mg or 500 mg Q3W. In one embodiment, vorasidenib is administered at a dose between about 10 mg/day and about 50 mg/day and pembrolizumab is administered at a dose of about 400 mg Q3W.

In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose between about 10 mg and about 500 mg every three weeks (Q3W) or every 6 weeks (Q6W). In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose between about 100 mg and about 400 mg Q3W or Q6W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose between about 200 mg and about 400 mg Q3W or Q6W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 100 mg, about 200 mg or about 400 mg Q3W or Q6W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 200 mg or about 400 mg Q3W or Q6W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose between about 100 mg and about 300 mg Q3W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 100 mg, 200 mg or 300 mg Q3W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 200 mg Q3W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose between about 200 mg and about 500 mg Q3W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 200 mg, 400 mg or 500 mg Q3W. In one embodiment, vorasidenib is administered at a dose between about 10 mg/day, about 15 mg/day, about 20 mg/day, about 25 mg/day, about 30 mg/day, about 35 mg/day, about 40 mg/day, about 45 mg/day or about 50 mg/day and pembrolizumab is administered at a dose of about 400 mg Q3W.

In one embodiment, vorasidenib is administered at a dose between about 10 mg/day, about 20 mg/day or about 40 mg/day and pembrolizumab is administered at a dose between about 10 mg and about 500 mg every three weeks (Q3W) or every 6 weeks (Q6W). In one embodiment, vorasidenib is administered at a dose between about 10 mg/day, about 20 mg/day or about 40 mg/day and pembrolizumab is administered at a dose between about 100 mg and about 400 mg Q3W or Q6W. In one embodiment, vorasidenib is administered at a dose between about 10 mg/day, about 20 mg/day or about 40 mg/day and pembrolizumab is administered at a dose between about 200 mg and about 400 mg Q3W or Q6W. In one embodiment, vorasidenib is administered at a dose between about 10 mg/day, about 20 mg/day or about 40 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg/day, about 20 mg/day or about 40 mg/day and pembrolizumab is administered at a dose of about 100 mg, about 200 mg or about 400 mg Q3W or Q6W. In one embodiment, vorasidenib is administered at a dose between about 10 mg/day, about 20 mg/day or about 40 mg/day and pembrolizumab is administered at a dose of about 200 mg or about 400 mg Q3W or Q6W. In one embodiment, vorasidenib is administered at a dose between about 10 mg/day, about 20 mg/day or about 40 mg/day and pembrolizumab is administered at a dose between about 100 mg and about 300 mg Q3W. In one embodiment, vorasidenib is administered at a dose between about 10 mg/day, about 20 mg/day or about 40 mg/day and pembrolizumab is administered at a dose of about 100 mg, 200 mg or 300 mg Q3W. In one embodiment, vorasidenib is administered at a dose between about 10 mg/day, about 20 mg/day or about 40 mg/day and pembrolizumab is administered at a dose of about 200 mg Q3W. In one embodiment, vorasidenib is administered at a dose between about 10 mg/day, about 20 mg/day or about 40 mg/day and pembrolizumab is administered at a dose between about 200 mg and about 500 mg Q3W. In one embodiment, vorasidenib is administered at a dose between about 10 mg/day, about 20 mg/day or about 40 mg/day and pembrolizumab is administered at a dose of about 200 mg, 400 mg or 500 mg Q3W. In one embodiment, vorasidenib is administered at a dose between about 10 mg/day, about 20 mg/day or about 40 mg/day and pembrolizumab is administered at a dose of about 400 mg Q3W.

In one embodiment, vorasidenib is administered at a dose between about 20 mg/day or about 40 mg/day and pembrolizumab is administered at a dose between about 10 mg and about 500 mg every three weeks (Q3W) or every 6 weeks (Q6W). In one embodiment, vorasidenib is administered at a dose between about 20 mg/day or about 40 mg/day and pembrolizumab is administered at a dose between about 100 mg and about 400 mg Q3W or Q6W. In one embodiment, vorasidenib is administered at a dose between about 20 mg/day or about 40 mg/day and pembrolizumab is administered at a dose between about 200 mg and about 400 mg Q3W or Q6W. In one embodiment, vorasidenib is administered at a dose between about 20 mg/day or about 40 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 20 mg/day or about 40 mg/day and pembrolizumab is administered at a dose of about 100 mg, about 200 mg or about 400 mg Q3W or Q6W. In one embodiment, vorasidenib is administered at a dose between about 20 mg/day or about 40 mg/day and pembrolizumab is administered at a dose of about 200 mg or about 400 mg Q3W or Q6W. In one embodiment, vorasidenib is administered at a dose between about 20 mg/day or about 40 mg/day and pembrolizumab is administered at a dose between about 100 mg and about 300 mg Q3W. In one embodiment, vorasidenib is administered at a dose between about 20 mg/day or about 40 mg/day and pembrolizumab is administered at a dose of about 100 mg, 200 mg or 300 mg Q3W. In one embodiment, vorasidenib is administered at a dose between about 20 mg/day or about 40 mg/day and pembrolizumab is administered at a dose of about 200 mg Q3W. In one embodiment, vorasidenib is administered at a dose between about 20 mg/day or about 40 mg/day and pembrolizumab is administered at a dose between about 200 mg and about 500 mg Q3W. In one embodiment, vorasidenib is administered at a dose between about 20 mg/day or about 40 mg/day and pembrolizumab is administered at a dose of about 200 mg, 400 mg or 500 mg Q3W. In one embodiment, vorasidenib is administered at a dose between about 20 mg/day or about 40 mg/day and pembrolizumab is administered at a dose of about 400 mg Q3W.

In one embodiment, vorasidenib is administered at a dose of about 40 mg/day and pembrolizumab is administered at a dose between about 10 mg and about 500 mg every three weeks (Q3W) or every 6 weeks (Q6W). In one embodiment, vorasidenib is administered at a dose of about 40 mg/day and pembrolizumab is administered at a dose between about 100 mg and about 400 mg Q3W or Q6W. In one embodiment, vorasidenib is administered at a dose of about 40 mg/day and pembrolizumab is administered at a dose between about 200 mg and about 400 mg Q3W or Q6W. In one embodiment, vorasidenib is administered at a dose of about 40 mg/day and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose of about 40 mg/day and pembrolizumab is administered at a dose of about 100 mg, about 200 mg or about 400 mg Q3W or Q6W. In one embodiment, vorasidenib is administered at a dose of about 40 mg/day and pembrolizumab is administered at a dose of about 200 mg or about 400 mg Q3W or Q6W. In one embodiment, vorasidenib is administered at a dose of about 40 mg/day and pembrolizumab is administered at a dose between about 100 mg and about 300 mg Q3W. In one embodiment, vorasidenib is administered at a dose of about 40 mg/day and pembrolizumab is administered at a dose of about 100 mg, 200 mg or 300 mg Q3W. In one embodiment, vorasidenib is administered at a dose of about 40 mg/day and pembrolizumab is administered at a dose of about 200 mg Q3W. In one embodiment, vorasidenib is administered at a dose of about 40 mg/day and pembrolizumab is administered at a dose between about 200 mg and about 500 mg Q3W. In one embodiment, vorasidenib is administered at a dose of about 40 mg/day and pembrolizumab is administered at a dose of about 200 mg, 400 mg or 500 mg Q3W. In one embodiment, vorasidenib is administered at a dose of about 40 mg/day and pembrolizumab is administered at a dose of about 400 mg Q3W.

In one embodiment, vorasidenib is administered at a dose between about 10 mg and about 100 mg once daily and pembrolizumab is administered at a dose between about 10 mg and about 500 mg every three weeks (Q3W) or every 6 weeks (Q6W). In one embodiment, vorasidenib is administered at a dose between about 10 mg and about 100 mg once daily and pembrolizumab is administered at a dose between about 100 mg and about 400 mg Q3W or Q6W. In one embodiment, vorasidenib is administered at a dose between about 10 mg and about 100 mg once daily and pembrolizumab is administered at a dose between about 200 mg and about 400 mg Q3W or Q6W. In one embodiment, vorasidenib is administered at a dose between about 10 mg and about 100 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg and about 100 mg once daily and pembrolizumab is administered at a dose of about 100 mg, about 200 mg or about 400 mg Q3W or Q6W. In one embodiment, vorasidenib is administered at a dose between about 10 mg and about 100 mg once daily and pembrolizumab is administered at a dose of about 200 mg or about 400 mg Q3W or Q6W. In one embodiment, vorasidenib is administered at a dose between about 10 mg and about 100 mg once daily and pembrolizumab is administered at a dose between about 100 mg and about 300 mg Q3W. In one embodiment, vorasidenib is administered at a dose between about 10 mg and about 100 mg once daily and pembrolizumab is administered at a dose of about 100 mg, 200 mg or 300 mg Q3W. In one embodiment, vorasidenib is administered at a dose between about 10 mg and about 100 mg once daily and pembrolizumab is administered at a dose of about 200 mg Q3W. In one embodiment, vorasidenib is administered at a dose between about 10 mg and about 100 mg once daily and pembrolizumab is administered at a dose between about 200 mg and about 500 mg Q3W. In one embodiment, vorasidenib is administered at a dose between about 10 mg and about 100 mg once daily and pembrolizumab is administered at a dose of about 200 mg, 400 mg or 500 mg Q3W. In one embodiment, vorasidenib is administered at a dose between about 10 mg and about 100 mg once daily and pembrolizumab is administered at a dose of about 400 mg Q3W.

In one embodiment, vorasidenib is administered at a dose between about 10 mg and about 50 mg once daily and pembrolizumab is administered at a dose between about 10 mg and about 500 mg every three weeks (Q3W) or every 6 weeks (Q6W). In one embodiment, vorasidenib is administered at a dose between about 10 mg and about 50 mg once daily and pembrolizumab is administered at a dose between about 100 mg and about 400 mg Q3W or Q6W. In one embodiment, vorasidenib is administered at a dose between about 10 mg and about 50 mg once daily and pembrolizumab is administered at a dose between about 200 mg and about 400 mg Q3W or Q6W. In one embodiment, vorasidenib is administered at a dose between about 10 mg and about 50 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg and about 50 mg once daily and pembrolizumab is administered at a dose of about 100 mg, about 200 mg or about 400 mg Q3W or Q6W. In one embodiment, vorasidenib is administered at a dose between about 10 mg and about 50 mg once daily and pembrolizumab is administered at a dose of about 200 mg or about 400 mg Q3W or Q6W. In one embodiment, vorasidenib is administered at a dose between about 10 mg and about 50 mg once daily and pembrolizumab is administered at a dose between about 100 mg and about 300 mg Q3W. In one embodiment, vorasidenib is administered at a dose between about 10 mg and about 50 mg once daily and pembrolizumab is administered at a dose of about 100 mg, 200 mg or 300 mg Q3W. In one embodiment, vorasidenib is administered at a dose between about 10 mg and about 50 mg once daily and pembrolizumab is administered at a dose of about 200 mg Q3W. In one embodiment, vorasidenib is administered at a dose between about 10 mg and about 50 mg once daily and pembrolizumab is administered at a dose between about 200 mg and about 500 mg Q3W. In one embodiment, vorasidenib is administered at a dose between about 10 mg and about 50 mg once daily and pembrolizumab is administered at a dose of about 200 mg, 400 mg or 500 mg Q3W. In one embodiment, vorasidenib is administered at a dose between about 10 mg and about 50 mg once daily and pembrolizumab is administered at a dose of about 400 mg Q3W.

In one embodiment, vorasidenib is administered at a dose between about 20 mg and about 40 mg once daily and pembrolizumab is administered at a dose between about 10 mg and about 500 mg every three weeks (Q3W) or every 6 weeks (Q6W). In one embodiment, vorasidenib is administered at a dose between about 20 mg and about 40 mg once daily and pembrolizumab is administered at a dose between about 100 mg and about 400 mg Q3W or Q6W. In one embodiment, vorasidenib is administered at a dose between about 20 mg and about 40 mg once daily and pembrolizumab is administered at a dose between about 200 mg and about 400 mg Q3W or Q6W. In one embodiment, vorasidenib is administered at a dose between about 20 mg and about 40 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 20 mg and about 40 mg once daily and pembrolizumab is administered at a dose of about 100 mg, about 200 mg or about 400 mg Q3W or Q6W. In one embodiment, vorasidenib is administered at a dose between about 20 mg and about 40 mg once daily and pembrolizumab is administered at a dose of about 200 mg or about 400 mg Q3W or Q6W. In one embodiment, vorasidenib is administered at a dose between about 20 mg and about 40 mg once daily and pembrolizumab is administered at a dose between about 100 mg and about 300 mg Q3W. In one embodiment, vorasidenib is administered at a dose between about 20 mg and about 40 mg once daily and pembrolizumab is administered at a dose of about 100 mg, 200 mg or 300 mg Q3W. In one embodiment, vorasidenib is administered at a dose between about 20 mg and about 40 mg once daily and pembrolizumab is administered at a dose of about 200 mg Q3W. In one embodiment, vorasidenib is administered at a dose between about 20 mg and about 40 mg once daily and pembrolizumab is administered at a dose between about 200 mg and about 500 mg Q3W. In one embodiment, vorasidenib is administered at a dose between about 20 mg and about 40 mg once daily and pembrolizumab is administered at a dose of about 200 mg, 400 mg or 500 mg Q3W. In one embodiment, vorasidenib is administered at a dose between about 20 mg and about 40 mg once daily and pembrolizumab is administered at a dose of about 400 mg Q3W.

In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose between about 10 mg and about 500 mg every three weeks (Q3W) or every 6 weeks (Q6W). In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose between about 100 mg and about 400 mg Q3W or Q6W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose between about 200 mg and about 400 mg Q3W or Q6W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 100 mg, about 200 mg or about 400 mg Q3W or Q6W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 200 mg or about 400 mg Q3W or Q6W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose between about 100 mg and about 300 mg Q3W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 100 mg, 200 mg or 300 mg Q3W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 200 mg Q3W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose between about 200 mg and about 500 mg Q3W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 200 mg, 400 mg or 500 mg Q3W. In one embodiment, vorasidenib is administered at a dose between about 10 mg, about 15 mg, about 20 mg, about 25 mg, about 30 mg, about 35 mg, about 40 mg, about 45 mg or about 50 mg once daily and pembrolizumab is administered at a dose of about 400 mg Q3W.

In one embodiment, vorasidenib is administered at a dose between about 10 mg, about 20 mg or about 40 mg once daily and pembrolizumab is administered at a dose between about 10 mg and about 500 mg every three weeks (Q3W) or every 6 weeks (Q6W). In one embodiment, vorasidenib is administered at a dose between about 10 mg, about 20 mg or about 40 mg once daily and pembrolizumab is administered at a dose between about 100 mg and about 400 mg Q3W or Q6W. In one embodiment, vorasidenib is administered at a dose between about 10 mg, about 20 mg or about 40 mg once daily and pembrolizumab is administered at a dose between about 200 mg and about 400 mg Q3W or Q6W. In one embodiment, vorasidenib is administered at a dose between about 10 mg, about 20 mg or about 40 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 10 mg, about 20 mg or about 40 mg once daily and pembrolizumab is administered at a dose of about 100 mg, about 200 mg or about 400 mg Q3W or Q6W. In one embodiment, vorasidenib is administered at a dose between about 10 mg, about 20 mg or about 40 mg once daily and pembrolizumab is administered at a dose of about 200 mg or about 400 mg Q3W or Q6W. In one embodiment, vorasidenib is administered at a dose between about 10 mg, about 20 mg or about 40 mg once daily and pembrolizumab is administered at a dose between about 100 mg and about 300 mg Q3W. In one embodiment, vorasidenib is administered at a dose between about 10 mg, about 20 mg or about 40 mg once daily and pembrolizumab is administered at a dose of about 100 mg, 200 mg or 300 mg Q3W. In one embodiment, vorasidenib is administered at a dose between about 10 mg, about 20 mg or about 40 mg once daily and pembrolizumab is administered at a dose of about 200 mg Q3W. In one embodiment, vorasidenib is administered at a dose between about 10 mg, about 20 mg or about 40 mg once daily and pembrolizumab is administered at a dose between about 200 mg and about 500 mg Q3W. In one embodiment, vorasidenib is administered at a dose between about 10 mg, about 20 mg or about 40 mg once daily and pembrolizumab is administered at a dose of about 200 mg, 400 mg or 500 mg Q3W. In one embodiment, vorasidenib is administered at a dose between about 10 mg, about 20 mg or about 40 mg once daily and pembrolizumab is administered at a dose of about 400 mg Q3W.

In one embodiment, vorasidenib is administered at a dose between about 20 mg or about 40 mg once daily and pembrolizumab is administered at a dose between about 10 mg and about 500 mg every three weeks (Q3W) or every 6 weeks (Q6W). In one embodiment, vorasidenib is administered at a dose between about 20 mg or about 40 mg once daily and pembrolizumab is administered at a dose between about 100 mg and about 400 mg Q3W or Q6W. In one embodiment, vorasidenib is administered at a dose between about 20 mg or about 40 mg once daily and pembrolizumab is administered at a dose between about 200 mg and about 400 mg Q3W or Q6W. In one embodiment, vorasidenib is administered at a dose between about 20 mg or about 40 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose between about 20 mg or about 40 mg once daily and pembrolizumab is administered at a dose of about 100 mg, about 200 mg or about 400 mg Q3W or Q6W. In one embodiment, vorasidenib is administered at a dose between about 20 mg or about 40 mg once daily and pembrolizumab is administered at a dose of about 200 mg or about 400 mg Q3W or Q6W. In one embodiment, vorasidenib is administered at a dose between about 20 mg or about 40 mg once daily and pembrolizumab is administered at a dose between about 100 mg and about 300 mg Q3W. In one embodiment, vorasidenib is administered at a dose between about 20 mg or about 40 mg once daily and pembrolizumab is administered at a dose of about 100 mg, 200 mg or 300 mg Q3W. In one embodiment, vorasidenib is administered at a dose between about 20 mg or about 40 mg once daily and pembrolizumab is administered at a dose of about 200 mg Q3W. In one embodiment, vorasidenib is administered at a dose between about 20 mg or about 40 mg once daily and pembrolizumab is administered at a dose between about 200 mg and about 500 mg Q3W. In one embodiment, vorasidenib is administered at a dose between about 20 mg or about 40 mg once daily and pembrolizumab is administered at a dose of about 200 mg, 400 mg or 500 mg Q3W. In one embodiment, vorasidenib is administered at a dose between about 20 mg or about 40 mg once daily and pembrolizumab is administered at a dose of about 400 mg Q3W.

In one embodiment, vorasidenib is administered at a dose of about 40 mg once daily and pembrolizumab is administered at a dose between about 10 mg and about 500 mg every three weeks (Q3W) or every 6 weeks (Q6W). In one embodiment, vorasidenib is administered at a dose of about 40 mg once daily and pembrolizumab is administered at a dose between about 100 mg and about 400 mg Q3W or Q6W. In one embodiment, vorasidenib is administered at a dose of about 40 mg once daily and pembrolizumab is administered at a dose between about 200 mg and about 400 mg Q3W or Q6W. In one embodiment, vorasidenib is administered at a dose of about 40 mg once daily and pembrolizumab is administered at a dose of about 100 mg, about 150 mg, about 200 mg, about 250 mg, about 300 mg, about 350 mg, about 400 mg, about 450 mg or about 500 mg Q3W or Q6W. In one embodiment, vorasidenib is administered at a dose of about 40 mg once daily and pembrolizumab is administered at a dose of about 100 mg, about 200 mg or about 400 mg Q3W or Q6W. In one embodiment, vorasidenib is administered at a dose of about 40 mg once daily and pembrolizumab is administered at a dose of about 200 mg or about 400 mg Q3W or Q6W. In one embodiment, vorasidenib is administered at a dose of about 40 mg once daily and pembrolizumab is administered at a dose between about 100 mg and about 300 mg Q3W. In one embodiment, vorasidenib is administered at a dose of about 40 mg once daily and pembrolizumab is administered at a dose of about 100 mg, 200 mg or 300 mg Q3W. In one embodiment, vorasidenib is administered at a dose of about 40 mg once daily and pembrolizumab is administered at a dose of about 200 mg Q3W. In one embodiment, vorasidenib is administered at a dose of about 40 mg once daily and pembrolizumab is administered at a dose between about 200 mg and about 500 mg Q3W. In one embodiment, vorasidenib is administered at a dose of about 40 mg once daily and pembrolizumab is administered at a dose of about 200 mg, 400 mg or 500 mg Q3W. In one embodiment, vorasidenib is administered at a dose of about 40 mg once daily and pembrolizumab is administered at a dose of about 400 mg Q3W.

Lower or higher doses than those recited above may be required. Specific dosage and treatment regimens for any particular subject depends upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination provided herein may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

### Methods of Use

The present invention provides a method of treatment of an enhancing brain tumor comprising administration to the patient of a novel therapeutic combination comprising vorasidenib and a humanized antibody against PD1 (e.g., pembrolizumab).

The present invention provides a method of treatment of enhancing glioma comprising administering to the patient a new combination comprising vorasidenib and a humanized antibody against PD1 (e.g., pembrolizumab).

In one embodiment, the brain tumors are recurrent or progressive.

In one embodiment, vorasidenib and pembrolizumab are administered concurrently. In one embodiment, vorasidenib and pembrolizumab are administered sequentially.

In one embodiment, vorasidenib and/or pembrolizumab can be administered repetitively, if necessary, for example, until the patient experiences stable disease or regression, or until the patient experiences disease progression or unacceptable toxicity. Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

### Brain Tumors Treated by Methods of the Invention

The methods of the invention are useful for treating brain tumors (e.g., enhancing brain tumors).

In some embodiments, the brain tumor is a glioma (e.g., diffuse adult glioma). In some embodiments, the brain tumor is an oligodendroglioma or astrocytoma. In some embodiments, the brain tumor is an oligodendroglioma. In some embodiments, the brain tumor is an astrocytoma.

In some embodiments, the brain tumor is an enhancing glioma (e.g., enhancing diffuse adult glioma). In some embodiments, the brain tumor is an enhancing oligodendroglioma or enhancing astrocytoma. In some embodiments, the brain tumor is an enhancing oligodendroglioma. In some embodiments, the brain tumor is an enhancing astrocytoma.

In some embodiments, the brain tumor is an enhancing recurrent or progressive glioma (e.g., enhancing recurrent or progressive diffuse adult glioma). In some embodiments, the brain tumor is an enhancing recurrent or progressive oligodendroglioma or enhancing recurrent or progressive astrocytoma. In some embodiments, the brain tumor is an enhancing recurrent or progressive oligodendroglioma. In some embodiments, the brain tumor is an enhancing recurrent or progressive astrocytoma.

In some embodiments, the brain tumor (e.g., glioma (e.g., astrocytoma)) to be treated is characterized by the presence of an IDH1 mutation, wherein the IDH1 mutation results in accumulation of R(−)-2-hydroxyglutarate in a patient. In one aspect of these embodiments, the IDH1 mutation results in accumulation of R(−)-2-hydroxyglutarate in a patient by providing a new ability of the enzyme to catalyze the NADPH-dependent reduction of α-ketoglutarate to R(−)-2-hydroxyglutarate in a patient. In another aspect of these embodiments, the IDH1 mutation is an R132X mutation. In another aspect of these embodiments, the R132X mutation is selected from R132H, R132C, R132L, R132V, R132S and R132G. In another aspect of these embodiments, the R132X mutation is R132H or R132C. In yet another aspect of these embodiments, the R132X mutation is R132H. In still another aspect of these embodiments, at least 30, 40, 50, 60, 70, 80 or 90% of the brain tumor (e.g., glioma (e.g., astrocytoma)) cells carry an IDH1 R132X mutation, such as an R132H, R132C, R132L, R132V, R132S or R132G mutation, at the time of diagnosis or treatment. A brain tumor (e.g., glioma (e.g., astrocytoma)) can be analyzed by sequencing cell samples to determine the presence and specific nature of (e.g., the changed amino acid present at) a mutation at amino acid 132 of IDH1.

In other embodiments, the brain tumor (e.g., glioma (e.g., astrocytoma)) to be treated is characterized by the presence of an IDH2 mutation, wherein the IDH2 mutation results in accumulation of R(−)-2-hydroxyglutarate in a patient. In one aspect of these embodiments, the IDH2 mutation results in accumulation of R(−)-2-hydroxyglutarate in a patient by providing a new ability of the enzyme to catalyze the NADPH-dependent reduction of α-ketoglutarate to R(−)-2-hydroxyglutarate in a patient. In another aspect of these embodiments, the mutant IDH2 has an R140X mutation. In another aspect of these embodiments, the R140X mutation is a R140Q mutation. In another aspect of these embodiments, the R140X mutation is a R140W mutation. In another aspect of these embodiments, the R140X mutation is a R140L mutation. In another aspect of these embodiments, the mutant IDH2 has an R172X mutation. In another aspect of these embodiments, the R172X mutation is a R172K mutation. In another aspect of these embodiments, the R172X mutation is a R172G mutation. In still another aspect of these embodiments, at least 30, 40, 50, 60, 70, 80 or 90% of the brain tumor (e.g., glioma (e.g., astrocytoma)) cells carry an IDH2 R140X and/or R172X mutation, such as an R140Q, R140W, or R140L and/or R172K or R172G mutation, at the time of diagnosis or treatment. A brain tumor (e.g., glioma (e.g., astrocytoma)) can be analyzed by sequencing cell samples to determine the presence and specific nature of (e.g., the changed amino acid present at) a mutation at amino acid 140 and/or 172 of IDH2.

In still other embodiments, the brain tumor (e.g., glioma (e.g., astrocytoma)) to be treated is characterized by the presence of an IDH1 mutation and an IDH2 mutation, wherein the IDH1 and IDH2 mutations collectively result in accumulation of R(−)-2-hydroxyglutarate in a patient. In one aspect of these embodiments, the IDH1 and IDH2 mutations result in accumulation of R(−)-2-hydroxyglutarate in a patient by providing a new ability of the enzyme to catalyze the NADPH-dependent reduction of α-ketoglutarate to R(−)-2-hydroxyglutarate in a patient. In various aspects of these embodiments, the IDH1 mutation is an R132X mutation selected from R132H, R132C, R132L, R132V, R132S and R132G. In various aspects of these embodiments, the IDH2 mutation is an R140Q, R140W, R140L, R172K or R172G mutation. In various other aspects of these embodiments, the brain tumor (e.g., glioma (e.g., astrocytoma)) to be treated is characterized by any combination of the foregoing IDH1 and IDH2 mutations. In still other aspects of these embodiments, at least 30, 40, 50, 60, 70, 80 or 90% of the brain tumor (e.g., glioma (e.g., astrocytoma)) cells carry an IDH1 R132X mutation, such as an R132H, R132C, R132L, R132V, R132S or R132G mutation, and an IDH2 R140X and/or R172X mutation, such as an R140Q, R140W, or R140L and/or R172K or R172G mutation, at the time of diagnosis or treatment. A brain tumor (e.g., glioma (e.g., astrocytoma)) can be analyzed by sequencing cell samples to determine the presence and specific nature of (e.g., the changed amino acid present at) a mutation at amino acid 132 of IDH1 and at amino acid 140 and/or 172 of IDH2.

In still other embodiments, the brain tumor (e.g., glioma (e.g., astrocytoma)) to be treated is characterized by the presence of an IDH1 allele that does not include an R132X mutation and an IDH2 allele that does not include an R140X or R172X mutation. In one aspect of these embodiments, at least 90% of the brain tumor (e.g., glioma (e.g., astrocytoma)) cells do not include a mutation at amino acid 132 of IDH1 or at amino acid 140 or 172 of IDH2 at the time of diagnosis or treatment. A brain tumor (e.g., glioma (e.g., astrocytoma)) can be analyzed by sequencing cell samples to determine the presence or absence of a mutation at amino acid 132 of IDH1 and at amino acid 140 and/or 172 of IDH2.

In one embodiment, the efficacy of treatment of the brain tumor is monitored by measuring the levels of 2HG in the subject. Typically, levels of 2HG are measured prior to treatment, wherein an elevated level is indicated for the use of the methods of treatment described herein. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy. Similarly, a determination that 2HG levels are not elevated during the course of or following treatment is also indicative of efficacy. Typically, 2HG measurements are utilized together with other well-known determinations of efficacy of brain tumor treatment, such as reduction in number and size of tumors and/or other cancer-associated lesions, improvement in the general health of the subject, and alterations in other biomarkers that are associated with brain tumor treatment efficacy.

2HG can be detected in a sample by the methods of PCT Publication No. WO 2011/050210 and US Publication No. US2012/0121515 hereby incorporated by reference in their entirety, or by analogous methods. In an exemplary method, 2HG can be detected in a sample by LC/MS. The sample is mixed 80:20 with methanol and centrifuged at 3,000 rpm for 20 minutes at 4 degrees Celsius. The resulting supernatant can be collected and stored at −80 degrees Celsius prior to LC-MS/MS to assess 2-hydroxyglutarate levels. A variety of different liquid chromatography (LC) separation methods can be used. Each method can be coupled by negative electrospray ionization (ESI, −3.0 kV) to triple-quadrupole mass spectrometers operating in multiple reaction monitoring (MRM) mode, with MS parameters optimized on infused metabolite standard solutions. Metabolites can be separated by reversed phase chromatography using 10 mM tributyl-amine as an ion pairing agent in the aqueous mobile phase, according to a variant of a previously reported method (Luo et al. J Chromatogr A 1147, 153-64, 2007). One method allows resolution of TCA metabolites: t=0, 50% B; t=5, 95% B; t=7, 95% B; t=8, 0% B, where B refers to an organic mobile phase of 100% methanol. Another method is specific for 2-hydroxyglutarate, running a fast linear gradient from 50%-95% B (buffers as defined above) over 5 minutes. A Synergi Hydro-RP, 100 mm×2 mm, 2.1 μm particle size (Phenomonex) can be used as the column, as described above. Metabolites can be quantified by comparison of peak areas with pure metabolite standards at known concentration. Metabolite flux studies from ¹³C-glutamine can be performed as described, e.g., in Munger et al. Nat Biotechnol 26, 1179-86, 2008.

In one embodiment, 2HG is directly evaluated.

In another embodiment, a derivative of 2HG formed in the process of performing the analytic method is evaluated. By way of example such a derivative can be a derivative formed in MS analysis. Derivatives can include a salt adduct, e.g., a Na adduct, a hydration variant, or a hydration variant which is also a salt adduct, e.g., a Na adduct, e.g., as formed in MS analysis. In another embodiment a metabolic derivative of 2HG is evaluated. Examples include species that build up or are elevated, or reduced, as a result of the presence of 2HG, such as glutarate or glutamate that will be correlated to 2HG, e.g., R-2HG.

Exemplary 2HG derivatives include dehydrated derivatives such as the compounds provided below, or a salt adduct thereof:

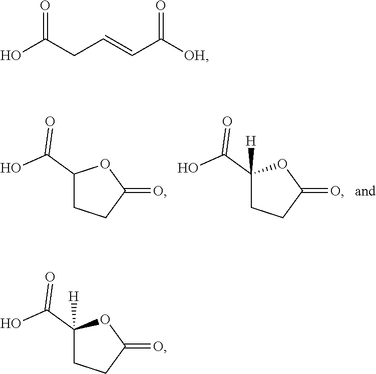

Treatment methods described herein can additionally comprise various evaluation steps prior to and/or following treatment with vorasidenib and pembrolizumab.

In one embodiment, prior to and/or after treatment with vorasidenib and pembrolizumab, the method further comprises the step of evaluating the growth, size, weight, invasiveness, stage and/or other phenotype of the brain tumor.

In one embodiment, prior to and/or after treatment with vorasidenib and pembrolizumab, the method further comprises the step of evaluating the IDH1 genotype of the brain tumor. This may be achieved by ordinary methods in the art, such as DNA sequencing, immuno analysis, and/or evaluation of the presence, distribution or level of 2HG.

In one embodiment, prior to and/or after treatment with vorasidenib and pembrolizumab, the method further comprises the step of determining the 2HG level in the subject. This may be achieved by spectroscopic analysis, e.g., magnetic resonance-based analysis, e.g., MRI and/or MRS measurement, sample analysis of bodily fluid, such as serum or spinal cord fluid analysis, or by analysis of surgical material, e.g., by mass-spectroscopy.

In an embodiment, provided herein is a method for treating a brain tumor in a subject in need thereof, comprising administering to the subject a therapeutically effective amount of vorasidenib, or a pharmaceutically acceptable acceptable salt, hydrate, solvate, or cocrystal thereof, and a therapeutically effective amount of pembrolizumab. In an embodiment, provided herein is a method for treating a brain tumor in a subject in need thereof, comprising administering to the subject vorasidenib at a dose between about 30 mg to 50 mg and pembrolizumab at a dose between about 150 mg to 250 mg. In an embodiment, provided herein is a method for treating a brain tumor in a subject in need thereof, comprising administering to the subject vorasidenib at a dose between about 30 mg/day to 50 mg/day and pembrolizumab at a dose between about 150 mg to 250 mg Q3W. In an embodiment, provided herein is a method for treating a brain tumor in a subject in need thereof, comprising administering to the subject vorasidenib at a dose between about 30 mg to 50 mg once daily and pembrolizumab at a dose between about 150 mg to 250 mg Q3W. In an embodiment, provided herein is a method for treating a brain tumor in a subject in need thereof, comprising administering to the subject vorasidenib at a dose between about 30 mg/day to 50 mg/day and pembrolizumab at a dose between about 150 mg to 250 mg Q6W. In an embodiment, provided herein is a method for treating a brain tumor in a subject in need thereof, comprising administering to the subject vorasidenib at a dose between about 30 mg to 50 mg once daily and pembrolizumab at a dose between about 150 mg to 250 mg Q6W. In an embodiment, provided herein is a method for treating a brain tumor in a subject in need thereof, comprising administering to the subject vorasidenib at a dose of about 40 mg and pembrolizumab at a dose of about 200 mg. In an embodiment, provided herein is a method for treating a brain tumor in a subject in need thereof, comprising administering to the subject vorasidenib at a dose of about 40 mg/day and pembrolizumab at a dose of about 200 mg Q3W. In an embodiment, provided herein is a method for treating a brain tumor in a subject in need thereof, comprising administering to the subject vorasidenib at a dose of about 40 mg once daily and pembrolizumab at a dose of about 200 mg Q3W. In an embodiment, provided herein is a method for treating a brain tumor in a subject in need thereof, comprising administering to the subject vorasidenib at a dose of about 40 mg/day and pembrolizumab at a dose of about 200 mg Q6W. In an embodiment, provided herein is a method for treating a brain tumor in a subject in need thereof, comprising administering to the subject vorasidenib at a dose of about 40 mg once daily and pembrolizumab at a dose of about 200 mg Q6W.

In an embodiment, provided herein is a method for treating a brain tumor in a subject in need thereof, comprising administering to the subject a therapeutically effective amount of vorasidenib, or a pharmaceutically acceptable salt, hydrate, solvate, or cocrystal thereof, and a therapeutically effective amount of pembrolizumab. In an embodiment, provided herein is a method for treating a brain tumor in a subject in need thereof, comprising administering to the subject vorasidenib at a dose between about 10 mg to 30 mg and pembrolizumab at a dose between about 150 mg to 250 mg. In an embodiment, provided herein is a method for treating a brain tumor in a subject in need thereof, comprising administering to the subject vorasidenib at a dose between about 10 mg/day to 30 mg/day and pembrolizumab at a dose between about 150 mg to 250 mg Q3W. In an embodiment, provided herein is a method for treating a brain tumor in a subject in need thereof, comprising administering to the subject vorasidenib at a dose between about 10 mg to 30 mg once daily and pembrolizumab at a dose between about 150 mg to 250 mg Q3W. In an embodiment, provided herein is a method for treating a brain tumor in a subject in need thereof, comprising administering to the subject vorasidenib at a dose between about 10 mg/day to 30 mg/day and pembrolizumab at a dose between about 150 mg to 250 mg Q6W. In an embodiment, provided herein is a method for treating a brain tumor in a subject in need thereof, comprising administering to the subject vorasidenib at a dose between about 10 mg to 30 mg once daily and pembrolizumab at a dose between about 150 mg to 250 mg Q6W. In an embodiment, provided herein is a method for treating a brain tumor in a subject in need thereof, comprising administering to the subject vorasidenib at a dose of about 20 mg once daily and pembrolizumab at a dose of about 200 mg Q3W. In an embodiment, provided herein is a method for treating a brain tumor in a subject in need thereof, comprising administering to the subject vorasidenib at a dose of about 20 mg/day and pembrolizumab at a dose of about 200 mg Q6W. In an embodiment, provided herein is a method for treating a brain tumor in a subject in need thereof, comprising administering to the subject vorasidenib at a dose of about 20 mg once daily and pembrolizumab at a dose of about 200 mg Q6W.

In an embodiment, provided herein is a method of treating astrocytoma in a subject in need thereof, comprising administering to the subject a therapeutically effective amount of vorasidenib, or a pharmaceutically acceptable salt, hydrate, solvate, or cocrystal thereof, and a therapeutically effective amount of pembrolizumab. In an embodiment, provided herein is a method of treating astrocytoma in a subject in need thereof, comprising administering to the subject vorasidenib at a dose between about 30 mg to 50 mg and pembrolizumab at a dose between about 150 mg to 250 mg. In an embodiment, provided herein is a method for treating astrocytoma in a subject in need thereof, comprising administering to the subject vorasidenib at a dose between about 30 mg/day to 50 mg/day and pembrolizumab at a dose between about 150 mg to 250 mg Q3W. In an embodiment, provided herein is a method for treating astrocytoma in a subject in need thereof, comprising administering to the subject vorasidenib at a dose between about 30 mg to 50 mg once daily and pembrolizumab at a dose between about 150 mg to 250 mg Q3W. In an embodiment, provided herein is a method for treating astrocytoma in a subject in need thereof, comprising administering to the subject vorasidenib at a dose between about 30 mg/day to 50 mg/day and pembrolizumab at a dose between about 150 mg to 250 mg Q6W. In an embodiment, provided herein is a method for treating astrocytoma in a subject in need thereof, comprising administering to the subject vorasidenib at a dose between about 30 mg to 50 mg once daily and pembrolizumab at a dose between about 150 mg to 250 mg Q6W. In an embodiment, provided herein is a method for treating astrocytoma in a subject in need thereof, comprising administering to the subject vorasidenib at a dose of about 40 mg and pembrolizumab at a dose of about 200 mg. In an embodiment, provided herein is a method for treating astrocytoma in a subject in need thereof, comprising administering to the subject vorasidenib at a dose of about 40 mg/day and pembrolizumab at a dose of about 200 mg Q3W. In an embodiment, provided herein is a method for treating astrocytoma in a subject in need thereof, comprising administering to the subject vorasidenib at a dose of about 40 mg once daily and pembrolizumab at a dose of about 200 mg Q3W. In an embodiment, provided herein is a method for treating astrocytoma in a subject in need thereof, comprising administering to the subject vorasidenib at a dose of about 40 mg/day and pembrolizumab at a dose of about 200 mg Q6W. In an embodiment, provided herein is a method for treating astrocytoma in a subject in need thereof, comprising administering to the subject vorasidenib at a dose of about 40 mg once daily and pembrolizumab at a dose of about 200 mg Q6W.

In an embodiment, provided herein is a method for treating astrocytoma in a subject in need thereof, comprising administering to the subject a therapeutically effective amount of vorasidenib, or a pharmaceutically acceptable salt, hydrate, solvate, or cocrystal thereof, and a therapeutically effective amount of pembrolizumab. In an embodiment, provided herein is a method for treating astrocytoma in a subject in need thereof, comprising administering to the subject vorasidenib at a dose between about 10 mg to 30 mg and pembrolizumab at a dose between about 150 mg to 250 mg. In an embodiment, provided herein is a method for treating astrocytoma in a subject in need thereof, comprising administering to the subject vorasidenib at a dose between about 10 mg/day to 30 mg/day and pembrolizumab at a dose between about 150 mg to 250 mg Q3W. In an embodiment, provided herein is a method for treating astrocytoma in a subject in need thereof, comprising administering to the subject vorasidenib at a dose between about 10 mg to 30 mg once daily and pembrolizumab at a dose between about 150 mg to 250 mg Q3W. In an embodiment, provided herein is a method for treating astrocytoma in a subject in need thereof, comprising administering to the subject vorasidenib at a dose between about 10 mg/day to 30 mg/day and pembrolizumab at a dose between about 150 mg to 250 mg Q6W. In an embodiment, provided herein is a method for treating astrocytoma in a subject in need thereof, comprising administering to the subject vorasidenib at a dose between about 10 mg to 30 mg once daily and pembrolizumab at a dose between about 150 mg to 250 mg Q6W. In an embodiment, provided herein is a method for treating astrocytoma in a subject in need thereof, comprising administering to the subject vorasidenib at a dose of about 20 mg once daily and pembrolizumab at a dose of about 200 mg Q3W. In an embodiment, provided herein is a method for treating astrocytoma in a subject in need thereof, comprising administering to the subject vorasidenib at a dose of about 20 mg/day and pembrolizumab at a dose of about 200 mg Q6W. In an embodiment, provided herein is a method for treating astrocytoma in a subject in need thereof, comprising administering to the subject vorasidenib at a dose of about 20 mg once daily and pembrolizumab at a dose of about 200 mg Q6W.

In an embodiment, provided herein is a method of treating astrocytoma with an IDH1 R132H mutation in a subject in need thereof, comprising administering to the subject a therapeutically effective amount of vorasidenib, or a pharmaceutically acceptable salt, hydrate, solvate, or cocrystal thereof, and a therapeutically effective amount of pembrolizumab. In an embodiment, provided herein is a method of treating astrocytoma with an IDH1 R132H mutation in a subject in need thereof, comprising administering to the subject vorasidenib at a dose between about 30 mg to 50 mg and pembrolizumab at a dose between about 150 mg to 250 mg. In an embodiment, provided herein is a method for treating astrocytoma with an IDH1 R132H mutation in a subject in need thereof, comprising administering to the subject vorasidenib at a dose between about 30 mg/day to 50 mg/day and pembrolizumab at a dose between about 150 mg to 250 mg Q3W. In an embodiment, provided herein is a method for treating astrocytoma with an IDH1 R132H mutation in a subject in need thereof, comprising administering to the subject vorasidenib at a dose between about 30 mg to 50 mg once daily and pembrolizumab at a dose between about 150 mg to 250 mg Q3W. In an embodiment, provided herein is a method for treating astrocytoma with an IDH1 R132H mutation in a subject in need thereof, comprising administering to the subject vorasidenib at a dose between about 30 mg/day to 50 mg/day and pembrolizumab at a dose between about 150 mg to 250 mg Q6W. In an embodiment, provided herein is a method for treating astrocytoma with an IDH1 R132H mutation in a subject in need thereof, comprising administering to the subject vorasidenib at a dose between about 30 mg to 50 mg once daily and pembrolizumab at a dose between about 150 mg to 250 mg Q6W. In an embodiment, provided herein is a method for treating astrocytoma with an IDH1 R132H mutation in a subject in need thereof, comprising administering to the subject vorasidenib at a dose of about 40 mg and pembrolizumab at a dose of about 200 mg. In an embodiment, provided herein is a method for treating astrocytoma with an IDH1 R132H mutation in a subject in need thereof, comprising administering to the subject vorasidenib at a dose of about 40 mg/day and pembrolizumab at a dose of about 200 mg Q3W. In an embodiment, provided herein is a method for treating astrocytoma with an IDH1 R132H mutation in a subject in need thereof, comprising administering to the subject vorasidenib at a dose of about 40 mg once daily and pembrolizumab at a dose of about 200 mg Q3W. In an embodiment, provided herein is a method for treating astrocytoma with an IDH1 R132H mutation in a subject in need thereof, comprising administering to the subject vorasidenib at a dose of about 40 mg/day and pembrolizumab at a dose of about 200 mg Q6W. In an embodiment, provided herein is a method for treating astrocytoma with an IDH1 R132H mutation in a subject in need thereof, comprising administering to the subject vorasidenib at a dose of about 40 mg once daily and pembrolizumab at a dose of about 200 mg Q6W.

In an embodiment, provided herein is a method for treating astrocytoma with an IDH1 R132H mutation in a subject in need thereof, comprising administering to the subject a therapeutically effective amount of vorasidenib, or a pharmaceutically acceptable salt, hydrate, solvate, or cocrystal thereof, and a therapeutically effective amount of pembrolizumab. In an embodiment, provided herein is a method for treating astrocytoma with an IDH1 R132H mutation in a subject in need thereof, comprising administering to the subject vorasidenib at a dose between about 10 mg to 30 mg and pembrolizumab at a dose between about 150 mg to 250 mg. In an embodiment, provided herein is a method for treating astrocytoma with an IDH1 R132H mutation in a subject in need thereof, comprising administering to the subject vorasidenib at a dose between about 10 mg/day to 30 mg/day and pembrolizumab at a dose between about 150 mg to 250 mg Q3W. In an embodiment, provided herein is a method for treating astrocytoma with an IDH1 R132H mutation in a subject in need thereof, comprising administering to the subject vorasidenib at a dose between about 10 mg to 30 mg once daily and pembrolizumab at a dose between about 150 mg to 250 mg Q3W. In an embodiment, provided herein is a method for treating astrocytoma with an IDH1 R132H mutation in a subject in need thereof, comprising administering to the subject vorasidenib at a dose between about 10 mg/day to 30 mg/day and pembrolizumab at a dose between about 150 mg to 250 mg Q6W. In an embodiment, provided herein is a method for treating astrocytoma with an IDH1 R132H mutation in a subject in need thereof, comprising administering to the subject vorasidenib at a dose between about 10 mg to 30 mg once daily and pembrolizumab at a dose between about 150 mg to 250 mg Q6W. In an embodiment, provided herein is a method for treating astrocytoma with an IDH1 R132H mutation in a subject in need thereof, comprising administering to the subject vorasidenib at a dose of about 20 mg once daily and pembrolizumab at a dose of about 200 mg Q3W. In an embodiment, provided herein is a method for treating astrocytoma with an IDH1 R132H mutation in a subject in need thereof, comprising administering to the subject vorasidenib at a dose of about 20 mg/day and pembrolizumab at a dose of about 200 mg Q6W. In an embodiment, provided herein is a method for treating astrocytoma with an IDH1 R132H mutation in a subject in need thereof, comprising administering to the subject vorasidenib at a dose of about 20 mg once daily and pembrolizumab at a dose of about 200 mg Q6W.

### EXAMPLES

### Example 1: A Phase 1, Safety Lead-In and Randomized, Open-Label, Perioperative Study of Vorasidenib in Combination with Pembrolizumab in Subjects with Recurrent or Progressive Enhancing IDH-1 Mutant Astrocytoma

This is a Phase 1, multi-center, safety lead-in and randomized, open-label, perioperative study of vorasidenib in combination with pembrolizumab in subjects with recurrent or progressive enhancing Grade 2 or 3 astrocytoma per World Health Organization 2016 criteria (i.e., absence of 1p19q co-deletion) with an IDH1 R132H mutation. The study is divided into two phases, a safety lead-in phase and a randomized perioperative phase.

### Safety Lead-In Phase

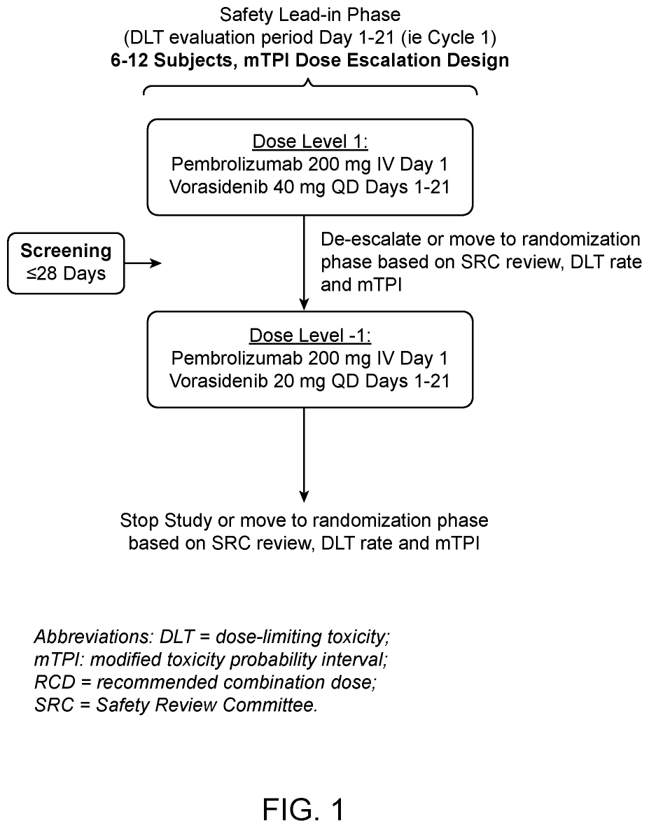

The safety lead-in phase is an open-label non-randomized part which will evaluate the safety and tolerability of vorasidenib in combination with pembrolizumab in order to determine the Recommended Combination Dose (RCD) of vorasidenib. This phase will enroll 6-12 subjects with recurrent or progressive Grade 2 or 3 astrocytoma with an IDH1 R132H mutation. The first cohort of subjects will receive a dose of 40 mg once daily (QD) of vorasidenib in combination with pembrolizumab 200 mg once every three weeks (Q3W). DLTs will be evaluated during the first 21 days of dosing, i.e., Cycle 1. Based on the DLT evaluation of 40 mg QD and the mTPI design, a second cohort may be opened to test an alternative dose of 20 mg QD. Following completion of the safety lead-in phase and determination of the vorasidenib RCD, the randomized perioperative phase will be opened. The design of the safety Lead-in Phase is shown in FIG. 1.

### Randomized Peri-Operative Phase

The randomized perioperative phase is an open-label randomized part which will evaluate CD3+ T-cell infiltration in tumors following pre-surgical treatment with vorasidenib in combination with pembrolizumab compared to untreated control tumors. Additional objectives include safety and tolerability, clinical activity, concentration of vorasidenib and 2-HG in tumors, and other immune cell infiltration in tumors. This phase will enroll approximately 60 subjects with Grade 2 or 3 enhancing astrocytomas with an IDH1-R132H mutation who are candidates for surgical resection.

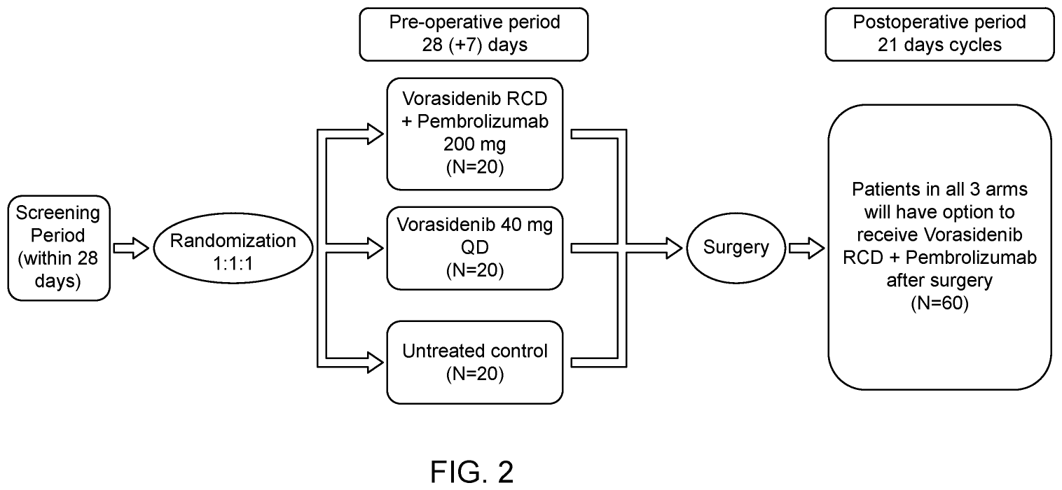

Subjects will be randomized in a 1:1:1 ratio to vorasidenib at the RCD in combination with pembrolizumab 200 mg Q3W, vorasidenib 40 mg QD only, or the untreated control group. There will be approximately 20 subjects per treatment group. Subjects randomized to receive a treatment will receive 28 (+7 days) of treatment prior to having a surgical resection. The design of the peri-operative phase is shown in FIG. 2.

Following surgery, all subjects will have the option to receive vorasidenib at the RCD in combination with pembrolizumab 200 mg Q3W. Subjects may continue to receive treatment until disease progression, unacceptable toxicity, or other discontinuation criteria are met. Subjects may continue to receive up to 35 cycles (approximately 24 months) of pembrolizumab 200 mg (this includes the 2 pembrolizumab doses in the pre-surgery period). Subjects who are still deriving clinical benefit after 35 cycles may continue to receive vorasidenib monotherapy until disease progression, unacceptable toxicity, or other discontinuation criteria are met. Postoperative tumor response assessments will be performed every 2 cycles up to and including Cycle 13 and every 4 cycles thereafter.

All subjects will be followed for overall survival (OS).

Having thus described several aspects of several embodiments, it is to be appreciated various alterations, modifications, and improvements will readily occur to those skilled in the art. Such alterations, modifications, and improvements are intended to be part of this disclosure and are intended to be within the spirit and scope of the invention. Accordingly, the foregoing description and drawings are by way of example only.

1. A method of treating an enhancing brain tumor in a patient in need thereof comprising administering to the patient a therapeutically effective amount of vorasidenib or a pharmaceutically acceptable salt, hydrate, solvate, or cocrystal thereof, and a therapeutically effective amount of pembrolizumab.

2. The method of claim 1, wherein the enhancing brain tumor is a glioma.

3. The method of claim 1, wherein the enhancing brain tumor is recurrent or progressive.

4. The method of claim 1, wherein the enhancing brain tumor is an oligodendroglioma or astrocytoma.

5. (canceled)

6. (canceled)

7. The method of claim 1, wherein the enhancing brain tumor is characterized by the presence of an IDH1 mutation, wherein the IDH1 mutation results in accumulation of R(−)-2-hydroxyglutarate in a patient.

8. The method of claim 7, wherein the IDH1 mutation is an R132X mutation.

9. The method of claim 8, wherein the IDH1 mutation is an R132H or R132C mutation.

10. The method of claim 1, wherein the enhancing brain tumor is characterized by the presence of an IDH2 mutation, wherein the IDH2 mutation results in accumulation of R(−)-2-hydroxyglutarate in a patient.

11. The method of claim 10, wherein the IDH2 mutation is an R140X mutation or an R172X mutation.

12. The method of claim 11, wherein the IDH2 mutation is an R140Q, R140W, R140L, R172K, or R172G mutation.

13. (canceled)

14. (canceled)

15. The method of claim 1, wherein the enhancing brain tumor is characterized by the presence of an IDH1 mutation and an IDH2 mutation, wherein the IDH1 and IDH2 mutations collectively result in accumulation of R(−)-2-hydroxyglutarate in a patient.

16. The method of claim 1, wherein vorasidenib is administered in non-salt form.

17. The method of claim 1, wherein vorasidenib is administered as a cocrystal.

18. The method of claim 17, wherein vorasidenib is administered as a cocrystal with citric acid.

19. The method of claim 1, wherein vorasidenib is administered at a dose between about 10 mg/day and about 100 mg/day, between about 10 mg/day and about 50 mg/day, or between about 20 mg/day and about 40 mg/day.

20. (canceled)

21. (canceled)

22. (canceled)

23. (canceled)

24. (canceled)

25. The method of claim 1, wherein vorasidenib is administered at a dose of about 40 mg/day.

26. The method of claim 1, wherein vorasidenib is administered once or twice daily.

27. (canceled)

28. (canceled)

29. (canceled)

30. (canceled)

31. (canceled)

32. (canceled)

33. (canceled)

34. (canceled)

35. The method of claim 1, wherein pembrolizumab is administered at a dose between about 10 mg and about 500 mg every three weeks (Q3W) or every 6 weeks (Q6W), between about 100 mg and about 400 mg Q3W or Q6W, or between about 200 mg and about 400 mg Q3W or Q6W.

36. (canceled)

37. (canceled)

38. The method of claim 1, wherein pembrolizumab is administered at a dose of about 100 mg, about 150 mg, about 200 mg, about 250 mg, about 300 mg, about 350 mg, about 400 mg, about 450 mg or about 500 mg Q3W or Q6W.

39. (canceled)

40. (canceled)

41. The method of claim 1, wherein pembrolizumab is administered Q3W.

42. The method of claim 1, wherein pembrolizumab is administered Q6W.

43. The method of claim 1, wherein pembrolizumab is administered at a dose between about 100 mg and about 300 mg Q3W.

44. (canceled)

45. The method of claim 1, wherein pembrolizumab is administered at a dose of about 200 mg Q3W.

46. The method of claim 1, wherein pembrolizumab is administered at a dose between about 200 mg and about 500 mg Q3W.

47. (canceled)

48. The method of claim 1, wherein pembrolizumab is administered at a dose of about 400 mg Q3W.

49. The method of claim 1, wherein vorasidenib and pembrolizumab are administered sequentially.

50. The method of claim 1, wherein vorasidenib and pembrolizumab are administered concurrently.
